(19) United States
(12) Patent Application Publication
(10) Pub. No.: US 2007/0248668 A1
(43) Pub. Date: Oct. 25, 2007

(54) Pharmaceutical compositions and uses thereof

(76) Inventors: Arthur F. Michaelis, Devon, PA (US); Panayiotis P. Constantinides, Gurnee, IL (US)

Correspondence Address: CLARK & ELBING LLP, 101 FEDERAL STREET, BOSTON, MA, 02110, US

(21) Appl. No.: 11/784,051

(22) Filed: Apr. 5, 2007

Related U.S. Application Data
(60) Provisional application No. 60/789,699, filed on Apr. 6, 2006.

(51) Int. Cl. A61K 31/5383; A61K 31/535; A61K 31/355; A61K 31/22; A61K 31/07

(52) U.S. Cl. 424/464; 514/229.5; 514/458; 514/474; 514/546; 514/725

(57) ABSTRACT

The invention features pharmaceutical compositions including rifalazil, a surfactant, and a lipophilic antioxidant and methods of use thereof.

### CROSS-REFERENCE TO RELATED APPLICATIONS

The application claims benefit of U.S. Provisional Application No. 60/789,699 filed Apr. 6, 2006, which is incorporated herein by reference.

### BACKGROUND OF THE INVENTION

The present invention relates to the field of antimicrobial therapy.

Rifalazil, an ansamycin-class antibiotic, has been described in U.S. Pat. No. 4,983,602.

A microgranulated formulation of rifalazil is disclosed in U.S. Pat. No. 5,547,683. This microgranulated rifalazil was shown to exhibit improved oral bioavailability in comparison to rifalazil crystals, mortar-milled crystals, and suspensions of mortar-milled crystals as determined by the relative AUCs produced for each formulation orally administered to beagles. Phase I clinical trials for rifalazil are described in U.S. Pat. Nos. 6,566,354 and 6,316,433.

A stable formulation for the oral administration of rifalazil that produces more consistent pharmacokinetics and an enhanced degree of bioavailability among subjects is desirable.

### SUMMARY OF THE INVENTION

We have discovered that the oral bioavailability of rifalazil is increased and the coefficient of variation in pharmacokinetic parameters (e.g., Cₘₐₓ and AUC_{∞}) is decreased when rifalazil is formulated with a sufficient amount of a surfactant. We have also discovered that the stability of such formulations is improved by addition of a lipophilic antioxidant.

Accordingly, in one aspect, the invention features a pharmaceutical composition for oral administration in unit dosage form including rifalazil, one or more surfactants, and a lipophilic antioxidant, wherein the surfactants are from 20% to 99% (w/w) of the composition.

In a related aspect, the invention features a pharmaceutical composition for oral administration in unit dosage form including rifalazil and an antioxidant surfactant. In certain embodiments, the antioxidant surfactant is retinyl palmitate, ascorbyl palmitate, or tocopheryl-PEG-1000-succinate.

The invention also features a pharmaceutical composition for oral administration in unit dosage form including rifalazil, a surfactant, and a lipophilic antioxidant, wherein the lipophilic antioxidant is present in an amount sufficient to reduce the oxidation of rifalazil. Desirably, upon storage of the unit dosage form at 25° C. and 60% relative humidity for a period of one month, six months, or even twelve months, less than 0.2% of the rifalazil is converted to rifalazil N-oxide. In certain embodiments, less than 0.2%, 0.15%, 0.10%, 0.05%, or 0.02% of the rifalazil is converted to rifalazil N-oxide upon storage of the unit dosage form at 25° C. and 60% relative humidity for a period of 1, 2, 3, 4, 5, 6, 7, 8, 9, 10, 11, 12, 18, or even 24 months.

In certain embodiments, the lipophilic antioxidant is selected, without limitation, from carotenoids, tocopherols and esters thereof, retinol and esters thereof, ascorbyl esters, butylhydroxyanisole (BHA), butylhydroxytoluene (BHT), propyl gallate, and mixtures thereof.

In one embodiment, the lipophilic antioxidant is an antioxidant surfactant, such as pegylated esters and fatty acid esters of tocopherol, retinol, ascorbic acid (e.g., retinyl palmitate, ascorbyl palmitate, and tocopheryl-PEG-1000-succinate), and mixtures thereof.

In one embodiment, the pharmaceutical composition includes from 1 to 50% (w/w) of a first lipophilic antioxidant selected from retinyl palmitate, ascorbyl palmitate, and tocopheryl-PEG-1000-succinate and less than 0.1% (w/w) of a second lipophilic antioxidant selected from tocopherol, tocopherol acetate, tocopherol nicotinoate, tocopherol succinate, tocotrienol, tocotrienol acetate, tocotrienol nicotinoate, tocotrienol succinate, carotenoids, BHT, BHA, and propylgallate. Desirably, the pharmaceutical composition includes from 1 to 20%, 1 to 15%, or 1 to 10% (w/w) of the first lipophilic antioxidant.

In another embodiment, the pharmaceutical composition further includes a hydrophilic co-solvent selected from alcohols (e.g., ethanol, propylene glycol, glycerol, and mixtures thereof), polyethylene glycols, and mixtures thereof. Desirably, the hydrophilic co-solvent is a polyethylene glycol with a molecular weight of between 200 and 10,000 Da. The hydrophilic co-solvent is combined with a surfactant, such as PEG-35 castor oil.

Pharmaceutical compositions of the invention combining both a hydrophilic polymer and a surfactant can include, for example, from 0.2 to 2.5% (w/w) rifalazil, from 75 to 85% (w/w) PEG-35 castor oil, from 0.5 to 1.5% (w/w) pluronic F68, from 8 to 15% PEG-400, from 1.5 to 2.5% (w/w) ascorbyl palmitate, from 0.01 to 0.05% (w/w) BHT, and from 1.5 to 2.5% (w/w) water.

Where the pharmaceutical composition of the invention contains a mixture of surfactants, it is desirable for the mixture to include at least one lipophilic surfactant (i.e., HLB<10) and at least one hydrophilic surfactant (i.e., HLB>10). For example, the pharmaceutical composition can include PEG-35 castor oil (HLB 12.5), PEG-8 caprylic/capric glycerides (Labrasol, HLB 14), and PEG-6 apricot kernel oil (Labrafil M1944, HLB 4).

Pharmaceutical compositions of the invention combining both a lipophilic surfactant and a hydrophilic surfactant can include, for example, from 0.2 to 2.5% (w/w) rifalazil, from 22 to 28% (w/w) PEG-35 castor oil, from 45 to 50% (w/w) PEG-6 apricot kernel oil, from 20 to 25% PEG-8 caprylic/capric glycerides, from 1.5 to 2.5% (w/w) ascorbyl palmitate, and from 0.01 to 0.05% (w/w) BHT.

In any of the above pharmaceutical compositions the solubility of rifalazil in the surfactants can be greater than 5 mg/mL. Desirably, the solubility is greater than 8 mg/mL, 10 mg/mL, 12 mg/mL, 14 mg/mL, 15 mg/mL, 16 mg/mL, 17 mg/mL, 18 mg/mL, 20 mg/mL, 22 mg/mL, 25 mg/mL, or 30 mg/mL.

The pharmaceutical compositions of the invention are in a unit dosage form. Desirably, the unit dosage form is a liquid-filled or semi-solid filled capsule (i.e., either as a hard capsule or a soft capsule). In the case of a hard capsule, the unit dosage form can also be a semi-solid-filled capsule. Capsule formulations of the invention are, desirably, greater than 20%, 30%, 40%, 50%, 60%, 70%, 80%, or 90% (w/w) one or more surfactants.

The pharmaceutical compositions of the invention can include a gelling agent (i.e., from 0.5 to 50%, 0.5 to 25%, 0.5 to 15%, 0.5 to 10%, 0.5 to 5%, or 0.5 to 3% (w/w) gelling agent) to increase the viscosity. Desirably, the gelling agent is a polyoxyethylene-polyoxypropylene block copolymer. These gelling agents are available under various trade names, including one or more of Synperonic PE series (ICI), Pluronic® series (BASF), Supronic, Monolan, Pluracare, and Plurodac. The generic term for these copolymers is “poloxamer” (CAS 9003-11-6). These polymers have the formula (I):
HO(C₂H₄O)ₐ(C₃H₆O)_{b}(C₂H₄O)ₐH (I)
where “a” and “b” denote the number of polyoxyethylene and polyoxypropylene units, respectively. These copolymers are available in molecular weights ranging from 1000 to 15000 daltons, and with ethylene oxide/propylene oxide ratios (a/b) between 0.1 and 3.0 by weight. Formulations of rifalazil according to the invention may include one or more of the polyoxyethylene-polyoxypropylene block copolymers above. In certain embodiments, the gelling agent is Pluronic® F68, also known as Poloxamer 188 in which a=75, b=30 (HLB=29).

Where the unit dosage formulation is a liquid or semi-solid-filled capsule, the formulation can include water to prevent dehydration of the capsule. Desirably, the capsule of rifalazil includes between 0.5% and 5%, 1% and 5%, 2% and 5%, 2% and 4%, or 2% and 3% (w/w) water.

Particular surfactants that may be used in the formulations described herein include polyethoxylated fatty acids, PEG-fatty acid diesters, PEG-fatty acid mono-ester and di-ester mixtures, polyethylene glycol glycerol fatty acid esters, alcohol-oil transesterification products, polyglycerized fatty acids, propylene glycol fatty acid esters, mixtures of propylene glycol esters and glycerol esters, mono- and diglycerides, sterol and sterol derivatives, polyethylene glycol sorbitan fatty acid esters, polyethylene glycol alkyl ethers, sugar esters, polyethylene glycol alkyl phenols, sorbitan fatty acid esters, lower alcohol fatty acid esters, polyoxyethylenes, and ionic surfactants. Any surfactant described herein may be used in the rifalazil formulations of the invention.

For any of the above pharmaceutical compositions, the composition can include between 0.5 and 100, 1 and 50, 1 and 30, 1 and 20, 1 and 15, 1 and 10, 1 and 5, or 2 and 20 mg of rifalazil. Desirably, the pharmaceutical composition contains about 1.0, 1.5, 2.0, 2.5, 3.0, 3.5, 4.0, 4.5, 5.0, 5.5, 10, 12.5, 15, 20, 25, or 30 mg of rifalazil.

For any of the above pharmaceutical compositions, the composition can include between 20% and 99%, 30% and 98%, 40% and 98%, 50% and 98%, 60% and 98%, or even 75% and 95% (w/w) surfactant.

For any of the pharmaceutical compositions of the invention, the surfactants are, desirably, present in an amount sufficient to produce, upon administration to fasted patients, a coefficient of variation in Cₘₐₓ of less than 60%. Desirably, the coefficient of variation in Cₘₐₓ is less than 55%, 50%, 45%, 40%, 35%, 30%, 25%, or even 20%.

For any of the pharmaceutical compositions of the invention, the surfactants are, desirably, present in an amount sufficient to produce, upon administration to fasted patients, a coefficient of variation in AUC_{∞} of less than 40%. Desirably, the coefficient of variation in AUC_{∞} is less than 35%, 30%, 25%, or even 20%.

For any of the pharmaceutical compositions of the invention, the surfactants are, desirably, present in an amount sufficient to produce, upon administration to fasted patients, a mean bioavailability of greater than 30%. Desirably, the mean bioavailability is greater than 35%, 40%, 45%, or even 50%.

The invention further features a method of treating a bacterial infection in a patient that includes the step of administering a rifalazil pharmaceutical composition of the invention, wherein the rifalazil is administered in an amount effective to treat the infection.

In any of the above methods, the infection is selected from community-acquired pneumonia, upper and lower respiratory tract infections, skin and soft tissue infections, hospital-acquired lung infections, bone and joint infections, respiratory tract infections, acute bacterial otitis media, bacterial pneumonia, urinary tract infections, complicated infections, noncomplicated infections, pyelonephritis, intra-abdominal infections, deep-seated abcesses, bacterial sepsis, central nervous system infections, bacteremia, wound infections, peritonitis, meningitis, infections after bum, urogenital tract infections, gastro-intestinal tract infections, pelvic inflammatory disease, endocarditis, and other intravascular infections. The methods of treating bacterial infections described herein are also useful in treating an infection is by a Gram-positive bacterium. Desirably, the methods are used to treat infection by a Gram-positive coccus, or by a drug-resistant Gram-positive coccus. Desirably, the Gram-positive coccus is selected from S. aureus, S. epidermidis, S. pneumoniae, S. pyogenes, M. catarrhalis, H. influenzae, and Enterococcus spp. Alternatively, the bacterial infection to be treated is by Chlamydia pneumoniae or Chlamydia trachomatis.

The methods of the invention can be used to reduce or eliminate the incidence of postoperative infections in patients undergoing surgical procedures or implantation of prosthetic devices.

The invention further features a method of treating an infection by multi-drug resistant bacteria in a patient. The method includes administering to the patient a rifalazil pharmaceutical composition of the invention, wherein the rifalazil is administered in an amount effective to treat the multi-drug resistant infection. Resistant strains of bacteria include penicillin-resistant, methicillin-resistant, quinolone-resistant, macrolide-resistant, and/or vancomycin-resistant bacterial strains. The multi-drug resistant bacterial infections to be treated using the methods of the invention include, for example, infections by penicillin-, methicillin-, macrolide-, vancomycin-, and/or quinolone-resistant Streptococcus pneumoniae; penicillin-, methicillin-, macrolide-, vancomycin-, and/or quinolone-resistant Staphylococcus aureus; penicillin-, methicillin-, macrolide-, vancomycin-, and/or quinolone-resistant Streptococcus pyogenes; and penicillin-, methicillin-, macrolide-, vancomycin-, and/or quinolone-resistant enterococci.

The invention also features a method of treating or preventing the development of an atherosclerosis-associated disease in a patient. The method includes administering to the patient (i) rifalazil and (ii) a lipophilic antioxidant simultaneously or within 14 days of each other in an amount, that together, is effective to treat or prevent the development of the atherosclerosis-associated disease in the patient. The patient is typically diagnosed as having the atherosclerosis-associated disease (or being at increased risk of developing the disease) or as having macrophages or foam cells infected with C. pneumoniae prior to the administration of a pharmaceutical composition of the invention.

The invention features a pharmaceutical composition including (i) rifalazil and (ii) a lipophilic antioxidant, wherein the rifalazil and the lipophilic antioxidant are each present in an amount that together is effective to treat an atherosclerosis-associated disease when administered to a patient.

The invention further features a kit including (i) a composition including rifalazil and a lipophilic antioxidant and (ii) instructions for administering the composition to a patient diagnosed with an atherosclerosis-associated disease.

The invention also features a kit including (i) rifalazil; and (ii) instructions for administering the rifalazil and a lipophilic antioxidant to a patient diagnosed with an atherosclerosis-associated disease.

In a certain embodiment of any of the above methods, compositions, and kits, the atherosclerosis-associated disease being treated is atherosclerosis or peripheral artery disease.

The invention also features a method of reducing the level of C-reactive protein in a patient in need thereof. This method includes administering to the patient (i) rifalazil and (ii) a lipophilic antioxidant simultaneously or within 14 days of each other in an amount, that together, is effective to reduce the level of C-reactive protein in the patient. In one embodiment, the patient has not been diagnosed as having a bacterial infection. In another embodiment, the patient has been diagnosed as having macrophages or foam cells infected with C. pneumoniae.

The invention also features a method for reducing C. pneumoniae replication in macrophages or foam cells in a patient in need thereof. This method includes administering to the patient a pharmaceutical composition of the invention, wherein the rifalazil is administered in an amount effective to reduce C. pneumoniae replication in macrophages or foam cells in the patient.

The invention also features a method for treating a persistent C. pneumoniae infection in macrophages or foam cells in a patient. The method includes administering to the patient a pharmaceutical composition of the invention, wherein the rifalazil is administered in an amount effective to treat the C. pneumoniae infection in macrophages or foam cells in the patient.

The invention also features a method for treating a chronic disease associated with an infection of C. pneumoniae. This method includes the step of administering to the patient a pharmaceutical composition of the invention, wherein the rifalazil is administered in an amount effective to treat the infection.

The invention features a method for treating a patient diagnosed as being infected with a bacterium having a multiplying form and a non-multiplying form by administering to the patient (i) a pharmaceutical composition of the invention, and (ii) a second antibiotic that is effective against the multiplying form of the bacterium, wherein the two antibiotics are administered in an amount and for a duration that together are effective to treat the infection.

In one preferred method of carrying out the foregoing method, the antibiotic that is effective against the multiplying form of the bacterium is administered in an amount and for a duration effective to reduce the number of bacteria in the patient to less than about 10⁶ organisms/mL. This typically takes from a few hours to 1, 2, or 3 days, but may take as long as a week. After this has been achieved, the patient is then administered a pharmaceutical composition of the invention, wherein the rifalazil is administered in an amount and for a duration effective to complete the treatment of the patient. Antibiotics that are effective against the multiplying form of the bacterium include any of the antibiotics described herein.

The invention also features a method of treating a patient diagnosed as having a chronic disease associated with a bacterial infection caused by bacteria capable of establishing a cryptic phase. The method includes the step of administering to the patient a pharmaceutical composition of the invention, wherein the rifalazil is administered in an amount effective to treat the patient.

The invention features a method of treating the cryptic phase of a bacterial infection. This method includes the step of administering to the patient a pharmaceutical composition of the invention. The administering is for a time and in an amount effective to treat the cryptic phase of the bacterial infection.

The invention features a method of treating a bacterial infection in a patient by (a) treating the multiplying form of the bacteria by administering an antibiotic to the patient for a time and an amount sufficient to treat the multiplying form, and (b) treating the non-multiplying form of the bacteria by administering a pharmaceutical composition of the invention, wherein the administering is for a time and in an amount effective to treat the non-multiplying form.

In any of the above methods, preferably, the bacterial infection is caused by one of the following: Chlamydia spp. (e.g., C. trachomatis, C. pneumoniae, C. psittaci, C. suis, C. pecorum, C. abortus, C. caviae, C. felis, C. muridarum), N. hartmannellae, W. chondrophila, S. negevensis, or P. acanthamoeba.

The time effective to treat a cryptic phase or other non-multiplying form of a bacterium ranges from one day to one year. In certain instances, treatment can be for several weeks or months, or even extended over the lifetime of the individual patient, if necessary. For example, the duration of treatment may be at least 30 days, at least 45 days, at least 90 days, or at least 180 days. Ultimately, it is most desirable to extend the treatment for such a time that the non-multiplying form is no longer detectable.

The invention also features a method for treating a patient having antibiotic-associated bacterial diarrhea or an infection of C. difficile, or preventing the disease or infection in the patient. The method includes the step of administering to the patient a pharmaceutical composition of the invention, wherein the rifalazil is administered in an amount effective to treat the infection. The method may be employed as an initial treatment of a patient having or being at risk for developing antibiotic-associated bacterial diarrhea or infection of C. difficile, or it may be employed to treat patients for whom the initial treatment (e.g., with metronidazole or vancomycin) has failed to fully treat the antibiotic-associated bacterial diarrhea or an infection of C. difficile. The method may be employed, for example, when the patient is colonized with C. difficile organisms that are resistant to one or more of metronidazole, vancomycin, and rifampicin.

The methods and compositions described herein can also be used to generate information useful, for example, for increasing investment in a company or increasing consumer demand for the methods and/or compositions.

The invention therefore features a method of increasing consumer demand for a pharmaceutical composition or therapeutic regimen described herein. The method includes the step of disseminating information about the pharmaceutical composition or therapeutic regimen.

The invention further features a method of increasing investment in a company seeking governmental approval for the sale of a pharmaceutical composition or therapeutic regimen described herein. The method includes the steps of i) disseminating information about the pharmaceutical composition or therapeutic regimen and ii) disseminating information about the intent of the company to market the pharmaceutical composition or therapeutic regimen.

Consumer demand for a pharmaceutical composition described herein, optionally with instructions to administer the pharmaceutical composition as part of a regimen described herein, can be increased by disseminating information about the utility, efficacy, or safety of the pharmaceutical composition or therapeutic regimen. Consumers include health maintenance organizations, hospitals, doctors, and patients. Typically, the information will be disseminated prior to a governmental approval for the sale of a composition or therapeutic regimen of the invention.

A company planning to sell a pharmaceutical composition described herein, optionally with instructions to administer the pharmaceutical composition as part of a regimen described herein, can increase investment therein by disseminating information about the company's intention to seek governmental approval for the sale of and disseminating information about the pharmaceutical composition or therapeutic regimen. For example, the company can increase investment by disseminating information about in vivo studies conducted, or planned, by the company, including, without limitation, information about the toxicity, efficacy, or dosing requirements of a pharmaceutical composition or therapeutic regimen of the invention. The company can also increase investment by disseminating information about the projected date of governmental approval of a pharmaceutical composition or therapeutic regimen of the invention.

Information can be disseminated in any of a variety of ways, including, without limitation, by press release, public presentation (e.g., an oral or poster presentation at a trade show or convention), on-line posting at a web site, and mailing. Information about the pharmaceutical composition or therapeutic regimen can include, without limitation, a structure, diagram, figure, chemical name, common name, tradename, formula, reference label, or any other identifier that conveys the identity of the pharmaceutical composition or therapeutic regimen of the invention to a person.

The compositions, methods, and kits of the invention may also apply to other rifamycins, including those described in U.S. Pat. Nos. 4,690,919; 4,983,602; 5,786,349; 5,981,522; 6,316,433 and 4,859,661, U.S. Patent Application Nos. 60/341,130 and 60/341,591, and U.S. Patent Publication Nos. US2005-0043298 A1; US2005-0137189 A1; and US2005-0197333 A1, each of which is hereby incorporated by reference.

By “in vivo studies” is meant any study in which a pharmaceutical composition or therapeutic regimen of the invention is administered to a mammal, including, without limitation, non-clinical studies, e.g., to collect data concerning toxicity and efficacy, and clinical studies.

By “projected date of governmental approval” is meant any estimate of the date on which a company will receive approval from a governmental agency to sell, e.g., to patients, doctors, or hospitals, a pharmaceutical composition or therapeutic regimen of the invention. A governmental approval includes, for example, the approval of a drug application by the Food and Drug Administration, among others.

As used herein, “bioavailability” refers to the fraction of drug absorbed following oral administration to a patient. Under fasted conditions the bioavailability of rifalazil formulated as described herein is at least 25%, but may be greater than 30%, 35%, 40%, 45%, or even 50% of the dose administered.

By “coefficient of variation” is meant the arithmetic standard deviation divided by the arithmetic mean for a particular pharmacokinetic parameter, wherein the data is obtained from a pharmacokinetic study involving 12 or more patients.

By “Cₘₐₓ” is meant the maximum concentration of rifalazil achieved in the blood after dosing.

By “AUC_{∞}” is meant the integrated area under the rifalazil plasma concentration versus time curve from t=0 to ∞.

By “food effect” is meant a difference between mean pharmacokinetic parameters Cₘₐₓ, Tₘₐₓ, AUC_{∞}, and bioavailability for rifalazil administered under fasted conditions in comparison to rifalazil administered under fed conditions.

As used herein, “reducing the food effect” refers to narrowing the difference between any one of Cₘₐₓ, Tₘₐₓ, AUC_{∞}, and bioavailability for rifalazil administered under fasted conditions in comparison to rifalazil administered under fed conditions, such that the differences are less than those observed for microgranulated rifalazil.

By “fed” or “fed conditions” is meant a subject has eaten within 30 minutes prior to drug administration.

By “fasted” or “fasted conditions” is meant a subject has not eaten for twelve hours prior and four hours subsequent to drug administration.

As used herein, the term “treating” refers to administering a pharmaceutical composition for prophylactic and/or therapeutic purposes. To “prevent disease” refers to prophylactic treatment of a patient who is not yet ill, but who is susceptible to, or otherwise at risk of, a particular disease. To “treat disease” or use for “therapeutic treatment” refers to administering treatment to a patient already suffering from a disease to improve or stabilize the patient's condition. Thus, in the claims and embodiments, treating is the administration to a patient either for therapeutic or prophylactic purposes.

By “patient” is meant a human.

As used herein, the term “administration” or “administering” refers to peroral administration of rifalazil to a patient.

As used herein, “an amount sufficient” refers to an amount of surfactant in a unit dosage formulation of rifalizil necessary to decrease the coefficient of variation in Cₘₐₓ, decrease the coefficient of variation in AUC_{∞}, reduce the food effect, or increase bioavailability in comparison to microgranulated rifalazil. The sufficient amount of surfactant used to practice the invention varies depending upon the amount of rifalazil in the unit dosage formulation and the nature of the surfactant or surfactant mixture. The sufficient amount can be determined by performing pharmacokinetic studies as described in Example 8.

The term “unit dosage form” refers to physically discrete units suitable as unitary dosages, such as a pill, tablet, caplet, hard capsule or soft capsule, each unit containing a predetermined quantity of rifalazil. The unit dosage forms of the invention include rifalazil and a surfactant.

By “hard capsule” is meant a capsule that includes a membrane that forms a two-part, capsule-shaped, container capable of carrying a solid, semi-solid, or liquid payload of drug and excipients.

By “soft capsule” is meant a capsule molded into a single container carrying a liquid payload of drug and excipients.

By “effective” amount is meant the amount of rifalazil required to treat or prevent an infection or a disease associated with an infection, such as peripheral artery disease. The effective amount of rifalazil used to practice the invention for therapeutic or prophylactic treatment of conditions caused by or contributed to by a microbial infection varies depending upon the manner of administration, the age, body weight, and general health of the subject. Ultimately, the attending physician will decide the appropriate amount and dosage regimen. Such amount is referred to as an “effective” amount.

As used in herein, a “surfactant” refers to any surface-active amphiphilic molecule, natural or synthetic. Surfactants can be amphiphilic molecules, e.g., molecules that are both oil- and water-soluble; lipophilic molecules, e.g., molecules that are soluble in oils, fats, and waxes; and hydrophilic molecules, e.g., molecules having an HLB value greater than 10 and are readily dispersable in water and other aqueous solvents. Surfactants include compounds that are micelle-forming, e.g., form aggregates in aqueous and biological fluids that are formed above certain surfactant concentration known as critical micelle concentration (CMC); compounds that form an emulsion in aqueous solutions, e.g., a colloidal dispersion of two immiscible liquids in the form of droplets, whose diameter, in general, is between 0.1 and 3.0 microns and which is typically optically opaque, unless the dispersed and continuous phases are refractive index matched; and compounds that form a microemulsion in aqueous solutions, e.g., a thermodynamically stable isotropically clear dispersion of two immiscible liquids, such as oil and water, stabilized by an interfacial film of surfactant molecules (i.e., a microemulsion has a mean droplet diameter of less than 200 nm, in general between 10-100 nm). The surfactants useful in the compositions and methods of the invention can be used as part of self-emulsifying drug delivery systems (SEDDS). Such systems include non-aqueous mixtures of oil(s) and surfactant(s), or lipophilic and hydrophilic surfactants as defined herein, with or without a co-solvent which form clear and isotropic solutions.

As used in herein, the term “lipophilic antioxidant” refers to a compound which (1) is at least partially soluble in the one or more surfactants present in the pharmaceutical compositions of the invention and (2) is capable, alone or in combination with another antioxidant, of reducing the oxidation of rifalazil when present in sufficient amounts in a formulation of the invention. Lipophilic antioxidants include, without limitation, tocopherols, tocotrienols, tocopherol acetate, tocopherol nicotinoate, tocopherol succinate, tocotrienol acetate, tocotrienol nicotinoate, tocotrienol succinate, retinol, carotenoids, butylhydroxyamide (BHA), butylhydroxytoluene (BHT), and propyl gallate, as well as compounds which are capable of functioning both as antioxidants and surfactants, such as pegylated tocopherols, pegylated retinols, and fatty acid esters of tocopherols, tocotrienols, retinol, and ascorbic acid. Preferred lipophilic antioxidants for use in the methods and compositions of the invention are tocopherol, tocopherol acetate, tocopherol nicotinoate, tocopherol succinate, tocotrienol, tocotrienol acetate, tocotrienol nicotinoate, tocotrienol succinate, carotenoids, butylhydroxyamide (BHA), butylhydroxytoluene (BHT), retinyl palmitate, ascorbyl palmitate, tocopheryl-PEG-1000-succinate (TPGS), and mixtures thereof.

As used herein, “carotenoid” refers to naturally-occurring pigments of the terpenoid group that can be found in plants, algae, bacteria, and certain animals, such as birds and shellfish. Carotenoids include carotenes, which are hydrocarbons (i.e., without oxygen), and their oxygenated derivatives (i.e., xanthophylls). Examples of carotenoids include lycopene; beta-carotene; zeaxanthin; echinenone; isozeaxanthin; astaxanthin; canthaxanthin; lutein; citranaxanthin; β-apo-8′-carotenic acid ethyl ester; hydroxy carotenoids, such as alloxanthin, apocarotenol, astacene, astaxanthin, capsanthin, capsorubin, carotenediols, carotenetriols, carotenols, cryptoxanthin, decaprenoxanthin, epilutein, fucoxanthin, hydroxycarotenones, hydroxyechinenones, hydroxylycopene, lutein, lycoxanthin, neurosporine, phytoene, phytofluoene, rhodopin, spheroidene, torulene, violaxanthin, and zeaxanthin; and carboxylic carotenoids, such as apocarotenoic acid, βapo-8′-carotenoic acid, azafrin, bixin, carboxylcarotenes, crocetin, diapocarotenoic acid, neurosporaxanthin, norbixin, and lycopenoic acid.

As used herein, the term “antioxidant surfactant” refers to compounds which function both as antioxidants and surfactants. Antioxidant surfactants include pegylated tocopherols, pegylated retinols, and fatty acid esters of tocopherols, tocotrienols, retinol, and ascorbic acid. Preferred antioxidant surfactants for use in the methods and compositions of the invention are retinyl palmitate, ascorbyl palmitate, tocopheryl-PEG-1000-succinate (TPGS), and mixtures thereof.

As used herein, the term “an amount sufficient to reduce the oxidation of rifalazil” refers to an amount of lipophilic antioxidant sufficient to reduce the amount of rifalazil N-oxide formed in a pharmaceutical composition of the invention upon storage for 4 weeks at 40° C. and 75% relative humidity (RH) in comparison to the same pharmaceutical composition formulated without a lipophilic antioxidant. The amount of rifalazil N-oxide formed upon storage of any pharmaceutical formulation of the invention can be determined by HPLC analysis as described in Example 8. Desirably, the lipophilic surfactant is present in an amount sufficient to reduce the amount of rifalazil N-oxide present at 4 weeks, 40° C., and 75% RH by 50%, 60%, 70%, 80%, 90%, 95%, 96%, 97%, 98%, 99%, or even %100 (i.e., below detectable limits) in comparison to the same pharmaceutical composition formulated without a lipophilic antioxidant.

As used herein, “HLB” values refer to the hydrophilic-lipophilic balance of a surfactant and defines the relative hydrophilicity and lipophilicity of the surfactants. Surfactants with lower HLB values are more lipophilic, and have greater solubility in oils, while surfactants with higher HLB values are more hydrophilic, and have greater solubility in aqueous solutions. For purposes of the present invention, surfactants having an HLB value less than 10 are “lipophilic surfactants,” while surfactants having an HLB value greater than 10 are “hydrophilic surfactants.” The HLB value derives from a semi-empirical formula used to index surfactants. Its value varies from 1-45 and in the case of non-ionic surfactants from about 1-20. The HLB system is based on the concept that some molecules have hydrophilic groups, other molecules have lipophilic groups, and some have both. Weight percentage of each type of group on a molecule or in a mixture predicts what behavior the molecular structure will exhibit. See, for example, Griffin, W C, J. Soc. Cos. Chem. 1:311 (1949); and Griffin, W C, J. Soc. Cos. Chem. 5:259 (1954). HLB values for exemplary surfactants which can be used in the methods and compositions of the invention are provided in Table 1, below.

TABLE 1

| Surfactant | HLB | Surfactant | HLB |
|---|---|---|---|
| PEG-2 Hydrogenated Castor Oil | 1.7 | PEG-10 oleyl ether | 12.4 |
| Sorbitan Trioleate | 1.8 | PEG-8 isooctylphenyl ether | 12.4 |
| Sorbitan Tristearate | 2.1 | PEG-10 stearyl ether | 12.4 |
| Glyceryl Stearate | 3.5 | PEG-35 Castor Oil | 12.5 |
| Sorbitan Sesquioleate | 3.7 | PEG-10 cetyl ether | 12.9 |
| Labrafil | 4.0 | Nonoxynol-9 | 12.9 |
| Sorbitan Oleate | 4.3 | PEG-40 Castor Oil | 13.0 |
| Sorbitan monostearate | 4.7 | PEG-10 isooctylphenyl ether | 13.5 |
| PEG-2 oleyl ether | 4.9 | PEG-40 Hydrogenated Castor Oil | 14.0 |
| PEG-2 stearyl ether | 4.9 | Labrasol | 14 |
| PEG-7 Hydrogenated Castor Oil | 5.0 | Nonoxynol-15 | 14.2 |
| PEG-2 cetyl ether | 5.3 | PEG-12 tridecyl ether | 14.5 |
| PEG-4 Sorbitan Stearate | 5.5 | PEG-18 tridecyl ether | 14.5 |
| PEG-2 Sorbitan Isostearate | 6.0 | Polysorbate 60 | 14.9 |
| Sorbitan Palmitate | 6.7 | Polysorbate 80 | 15.0 |
| Triton SP-135 | 8.0 | PEG-20 Glyceryl Stearate | 15.0 |
| Sorbitan monolaurate | 8.6 | PEG-20 Stearate | 15.0 |
| PEG-40 Sorbitan Peroleate | 9.5 | PEG-20 stearyl ether | 15.3 |
| PEG-4 lauryl ether | 9.7 | PEG-20 oleyl ether | 15.3 |
| Polysorbate 81 | 10.0 | Polysorbate 40 | 15.6 |
| PEG-40 Sorbitan Hexaoleate | 10.0 | PEG20 cetyl ether | 15.7 |
| PEG-40 Sorbitan Perisostearate | 10.0 | PEG(20) hexadecyl ether | 15.7 |
| PEG-10 Olive Glycerides | 10.0 | PEG-60 Hydrogenated Castor Oil | 16.0 |
| PEG sorbitol hexaoleate | 10.2 | PEG-30 Stearate | 16.5 |
| Polysorbate 65 | 10.5 | Polysorbate 20 | 16.7 |
| PEG-25 Hydrogenated Castor Oil | 10.8 | PEG-75 Lanolin | 16.7 |
| Polysorbate 85 | 11.0 | PEG23 lauryl ether | 16.9 |
| PEG-7 Glyceryl Cocoate | 11.0 | PEG-40 Stearate | 17.3 |
| PEG-8 Stearate | 11.1 | PEG-50 Stearate | 17.7 |
| PEG sorbitan tetraoleate | 11.4 | PEG40 isooctylphenyl ether | 17.9 |
| PEG-15 Glyceryl Isostearate | 12.0 | PEG-100 Stearate | 18.8 |
| PEG-35 Almond Glycerides | 12.0 | Pluronic F68 | 29.0 |

By “bacterial infection” is meant the invasion of a host by pathogenic bacteria. For example, the infection may include the excessive growth of bacteria that are normally present in or on the body of a human or growth of bacteria that are not normally present in or on a human. More generally, a bacterial infection can be any situation in which the presence of a bacterial population(s) is damaging to a host body. Thus, a human is “suffering” from a bacterial infection when an excessive amount of a bacterial population is present in or on the person's body, or when the presence of a bacterial population(s) is damaging the cells or other tissue of the person.

By “atherosclerosis” is meant the progressive accumulation of smooth muscle cells, immune cells (e.g., lymphocytes, macrophages, or monocytes), lipid products (e.g., lipoproteins, or cholesterol), cellular waste products, calcium, or other substances within the inner lining of an artery, resulting in the narrowing or obstruction of the blood vessel and the development of atherosclerosis-associated diseases. Atherosclerosis is typically manifested within large and medium-sized arteries, and is often characterized by a state of chronic inflammation within the arteries.

By “atherosclerosis-associated disease” is meant any disorder that is caused by or is associated with atherosclerosis, particularly peripheral artery disease. Typically, atherosclerosis of the coronary arteries commonly causes coronary artery disease, myocardial infarction, coronary thrombosis, and angina pectoris. Atherosclerosis of the arteries supplying the central nervous system frequently provokes strokes and transient cerebral ischemia. In the peripheral circulation, atherosclerosis causes intermittent claudication and gangrene and can jeopardize limb viability. Atherosclerosis of an artery of the splanchnic circulation can cause mesenteric ischemia. Atherosclerosis can also affect the kidneys directly (e.g., renal artery stenosis).

A patient who is being treated for an atherosclerosis-associated disease is one who a medical practitioner has diagnosed as having such a disease. Diagnosis may be by any suitable means. Methods for diagnosing atherosclerosis by measuring systemic inflammatory markers are described, for example, in U.S. Pat. No. 6,040,147, hereby incorporated by reference. Diagnosis and monitoring may employ an electrocardiogram, chest X-ray, echocardiogram, cardiac catheterization, ultrasound (for the measurement of vessel wall thickness), or measurement of blood levels of CPK, CPK-MB, myoglobin, troponin, homocysteine, or C-reactive protein. A patient in whom the development of an atherosclerosis-associated disease is being prevented is one who has not received such a diagnosis. One in the art will understand that these patients may have been subjected to the same tests (electrocardiogram, chest X-ray, etc.) or may have been identified, without examination, as one at high risk due to the presence of one or more risk factors (e.g., family history, hypertension, diabetes mellitus, high cholesterol levels). Thus, prophylactic administration of a pharmaceutical composition of the invention is considered to be preventing the development of an atherosclerosis-associated disease.

By “peripheral artery disease” is meant the progressive accumulation of smooth muscle cells, immune cells (e.g., lymphocytes, macrophages, or monocytes), lipid products (e.g., lipoproteins, or cholesterol), cellular waste products, calcium, or other substances within the inner lining of an artery, resulting in the restriction of blood circulation, mainly in arteries leading to the kidneys, stomach, arms, legs and feet. In its early stages a common symptom is cramping or fatigue in the legs and buttocks during activity.

An atherosclerosis-associated disease has been treated or prevented when one or more tests of the disease (e.g., any of those described above) indicate that the patient's condition has improved or the patient's risk reduced. In one example, a reduction in C-reactive protein to normal levels indicates that an atherosclerosis-associated disease has been treated or prevented.

An alternative means by which treatment or prevention is assessed includes determination of the presence of an infection of C. pneumoniae. Any suitable method may be employed (e.g., determination of C. pneumoniae in blood monocytes or in the atheroma itself (e.g., in macrophages or foam cells present in the fatty streak), or detection of C. pneumoniae DNA, RNA, or antibodies to C. pneumoniae in a biological sample from the patient).

“Antibiotic-associated bacterial diarrhea” means the condition wherein antibiotic therapy disturbs the balance of the microbial flora of the gut, allowing pathogenic organisms such as C. difficile to flourish. These organisms cause diarrhea. Antibiotic-associated bacterial diarrhea includes such conditions as C. difficile associated diarrhea (CDAD) and pseudomembranous colitis. When a pharmaceutical composition of the invention is administered for the treatment of a C. difficile infection, an effective amount of rifalazil is the amount required to eradicate C. difficile from the patient, or the amount which prevents an infection of C. difficile, as determined by a diagnostic test that detects C. difficile.

“Pseudomembranous colitis,” also known as pseudomembranous enterocolitis or enteritis, means the inflammation of the mucous membrane of both small and large intestine with the formation and passage of pseudomembranous material (composed of fibrin, mucous, necrotic epithelial cells and leukocytes) in the stools.

The term “lower gastrointestinal tract” means the lower part of the small intestine (ileum) and the colon.

By “autoimmune disease” is meant a disease arising from an immune reaction against self-antigens and directed against the individual's own tissues. Examples of autoimmune diseases include but are not limited to systemic lupus erythematosus, rheumatoid arthritis, myasthenia gravis, and Graves' disease.

By “bacteria” is meant a unicellular prokaryotic microorganism that usually multiplies by cell division.

By “bacteria capable of establishing a cryptic phase” is meant any species whose life cycle includes a persistent, non-multiplying phase. These species include but are not limited to C. trachomatis, C. pneumoniae, C. psittaci, C. suis, C. pecorum, C. abortus, C. caviae, C. felis, C. muridarum, N. hartmannellae, W. chondrophila, S. negevensis, and P. acanthamoeba, as well as any other species described in Everett et al. (Int. J. Syst. Evol. Microbiol. 49:415-440 (1999)).

By “chronic disease” is meant an inveterate disease of long continuance, or which progresses slowly, in contrast to an acute disease, which rapidly terminates. A chronic disease may begin with a rapid onset or in a slow insidious manner but it tends to persist for several weeks, months or years, and has a vague and indefinite termination.

By “cryptic phase” is meant the latent or dormant intracellular phase of infection characterized by little or no metabolic activity. The non-replicating cryptic phase is often characteristic of persistent forms of intracellular bacterial infections.

By “elementary body phase” is meant the infectious phase of the bacterial life cycle which is characterized by the presence of elementary bodies (EBs). EBs are small (300-400 nm), infectious, spore-like forms which are metabolically inactive, non-replicating, and found most often in the acellular milieu. EBs possess a rigid outer membrane which protects them from a variety of physical insults such as enzymatic degradation, sonication and osmotic pressure.

By “immunocompromised” is meant a person who exhibits an attenuated or reduced ability to mount a normal cellular or humoral defense to challenge by infectious agents, e.g., viruses, bacterial, fungi, and protozoa. Persons considered immunocompromised include malnourished patients, patients undergoing surgery and bone narrow transplants, patients undergoing chemotherapy or radiotherapy, neutropenic patients, HIV-infected patients, trauma patients, bum patients, patients with chronic or resistant infections such as those resulting from myelodysplastic syndrome, and the elderly, all of who may have weakened immune systems.

By “inflammatory disease” is meant a disease state characterized by (1) alterations in vascular caliber that lead to an increase in blood flow, (2) structural changes in the microvasculature that permit the plasma proteins and leukocytes to leave the circulation, and (3) emigration of the leukocytes from the microcirculation and their accumulation in the focus of injury. The classic signs of acute inflammation are erythema, edema, tenderness (hyperalgesia), and pain. Chronic inflammatory diseases are characterized by infiltration with mononuclear cells (e.g., macrophages, lymphocytes, and plasma cells), tissue destruction, and fibrosis. Non-limiting examples of inflammatory disease include asthma, coronary artery disease, arthritis, conjunctivitis, lymphogranuloma venerum, and salpingitis.

By “intracytoplasmic inclusion” is meant a replicating reticulate body (RB) that has no cell wall. Such inclusions may be detected, for example, through chlamydiae sample isolation and propagation on a mammalian cell lines, followed by fixing and staining using one of a variety of staining methods including Giemsa staining, iodine staining, and immunofluorescence. These inclusions have a typical round or oval appearance.

By “persistent bacterial infection” is meant an infection that is not completely eradicated through standard treatment regimens using antibiotics. Persistent bacterial infections are caused by bacteria capable of establishing a cryptic phase or other non-multiplying form of a bacterium and may be classified as such by culturing bacteria from a patient and demonstrating bacterial survival in vitro in the presence of antibiotics or by determination of anti-bacterial treatment failure in a patient. As used herein, a persistent infection in a patient includes any recurrence of an infection, after receiving antibiotic treatment, from the same species more than two times over the period of two or more years or the detection of the cryptic phase of the infection in the patient. An in vivo persistent infection can be identified through the use of a reverse transcriptase polymerase chain reaction (RT-PCR) to demonstrate the presence of 16S rRNA transcripts in bacterially infected cells after treatment with one or more antibiotics (Antimicrob. Agents Chemother. 12:3288-3297 (2000)).

As used herein, “non-multiplying” phase or bacteria refers to the non-multiplying growth phase of bacteria. Typically, the non-multiplying bacteria will survive standard antimicrobial therapy (see, e.g., Martinez et al., Antimicrob. Agents Chemother. 44:1771-1777 (2000); Riesenfeld et al., Antimicrob. Agents Chemother. 41:2059-2060 (1997); Alonso et al., Microbiology 145:2857-2862 (1999)).

By “replicating phase” is meant the phase of the bacterial cell cycle characterized by the presence of an RB. The RB is the actively replicating form of the Chlamydia. It contains no cell wall and is detected as an inclusion in the cell.

The term “microbial infection” refers to the invasion of the host patient by pathogenic microbes. This includes the excessive growth of microbes that are normally present in or on the body of a patient. More generally, a microbial infection can be any situation in which the presence of a microbial population(s) is damaging to a host patient. Thus, a patient is “suffering” from a microbial infection when excessive numbers of a microbial population are present in or on a patient's body, or when the presence of a microbial population(s) is damaging the cells or other tissue of a patient.

When administered to a human, pharmaceutical compositions described herein can provide an increase in the bioavailability of rifalazil in comparison to the administration of microgranulated rifalazil disclosed in U.S. Pat. No. 5,547,683. The rifalazil formulations also decrease the coefficient of variation in pharmacokinetic parameters (e.g., Cₘₐₓ and AUC_{∞}) in comparison to the microgranulated formulation.

### DETAILED DESCRIPTION

The invention provides stable pharmaceutical formulations including rifalazil, a surfactant, and a lipophilic antioxidant. The formulations are useful for decreasing the coefficient of variation in Cₘₐₓ, decreasing the coefficient of variation in AUC_{∞}, reducing the food effect, and/or increasing the bioavailability of rifalazil.

Formulation

As described herein, surfactants can be added to rifalazil in a unit dosage form for oral administration. The excipients can increase the solubilization of rifalazil in the gut, increasing overall rifalazil absorption and reducing the variability in the PK parameters observed in a patient population. The excipients used are restricted to those that have a high degree of safety in humans.

A variety of surfactants may be used for the formulation of rifalazil including those disclosed in U.S. Pat. No. 6,365,637, incorporated herein by reference and compounds belonging to the following classes: polyethoxylated fatty acids, PEG-fatty acid diesters, PEG-fatty acid mono-ester and di-ester mixtures, polyethylene glycol glycerol fatty acid esters, alcohol-oil transesterification products, polyglycerized fatty acids, propylene glycol fatty acid esters, mixtures of propylene glycol esters and glycerol esters, mono- and diglycerides, sterol and sterol derivatives, polyethylene glycol sorbitan fatty acid esters, polyethylene glycol alkyl ethers, sugar esters, polyethylene glycol alkyl phenols, sorbitan fatty acid esters, lower alcohol fatty acid esters, polyoxyethylenes, and ionic surfactants. Commercially available examples for each class of excipient are provided below.

Polyethoxylated fatty acids may be used as excipients for the formulation of rifalazil. Examples of commercially available polyethoxylated fatty acid monoester surfactants include: PEG 4-100 monolaurate (Crodet L series, Croda), PEG 4-100 monooleate (Crodet O series, Croda), PEG 4-100 monostearate (Crodet S series, Croda, and Myrj Series, Atlas/ICI), PEG 400 distearate (Cithrol 4DS series, Croda), PEG 100, 200, or 300 monolaurate (Cithrol ML series, Croda), PEG 100, 200, or 300 monooleate (Cithrol MO series, Croda), PEG 400 dioleate (Cithrol 4DO series, Croda), PEG 400-1000 monostearate (Cithrol MS series, Croda), PEG-1 stearate (Nikkol MYS-1EX, Nikko, and Coster K1, Condea), PEG-2 stearate (Nikkol MYS-2, Nikko), PEG-2 oleate (Nikkol MYO-2, Nikko), PEG-4 laurate (Mapeg® 200 ML, PPG), PEG-4 oleate (Mapeg® 200 MO, PPG), PEG-4 stearate (Kessco® PEG 200 MS, Stepan), PEG-5 stearate (Nikkol TMGS-5, Nikko), PEG-5 oleate (Nikkol TMGO-5, Nikko), PEG-6 oleate (Algon OL 60, Auschem SpA), PEG-7 oleate (Algon OL 70, Auschem SpA), PEG-6 laurate (Kessco® PEG300 ML, Stepan), PEG-7 laurate (Lauridac 7, Condea), PEG-6 stearate (Kessco® PEG300 MS, Stepan), PEG-8 laurate (Mapeg® 400 ML, PPG), PEG-8 oleate (Mapeg® 400 MO, PPG), PEG-8 stearate (Mapeg® 400 MS, PPG), PEG-9 oleate (Emulgante A9, Condea), PEG-9 stearate (Cremophor S9, BASF), PEG-10 laurate (Nikkol MYL-10, Nikko), PEG-10 oleate (Nikkol MYO-10, Nikko), PEG-12 stearate (Nikkol MYS-10, Nikko), PEG-12 laurate (Kessco® PEG 600 ML, Stepan), PEG-12 oleate (Kessco® PEG 600 MO, Stepan), PEG-12 ricinoleate (CAS # 9004-97-1), PEG-12 stearate (Mapeg® 600 MS, PPG), PEG-15 stearate (Nikkol TMGS-15, Nikko), PEG-15 oleate (Nikkol TMGO-15, Nikko), PEG-20 laurate (Kessco® PEG 1000 ML, Stepan), PEG-20 oleate (Kessco® PEG 1000 MO, Stepan), PEG-20 stearate (Mapeg® 1000 MS, PPG), PEG-25 stearate (Nikkol MYS-25, Nikko), PEG-32 laurate (Kessco® PEG 1540 ML, Stepan), PEG-32 oleate (Kessco® PEG 1540 MO, Stepan), PEG-32 stearate (Kessco® PEG 1540 MS, Stepan), PEG-30 stearate (Myrj 51), PEG-40 laurate (Crodet L40, Croda), PEG-40 oleate (Crodet O40, Croda), PEG-40 stearate (Emerest® 2715, Henkel), PEG-45 stearate (Nikkol MYS-45, Nikko), PEG-50 stearate (Myrj 53), PEG-55 stearate (Nikkol MYS-55, Nikko), PEG-100 oleate (Crodet O-100, Croda), PEG-100 stearate (Ariacel 165, ICI), PEG-200 oleate (Albunol 200 MO, Taiwan Surf.), PEG-400 oleate (LACTOMUL, Henkel), and PEG-600 oleate (Albunol 600 MO, Taiwan Surf.). Formulations of rifalazil according to the invention may include one or more of the polyethoxylated fatty acids above.

Polyethylene glycol fatty acid diesters may also be used as excipients for the formulation of rifalazil. Examples of commercially available polyethylene glycol fatty acid diesters include: PEG-4 dilaurate (Mapeg® 200 DL, PPG), PEG-4 dioleate (Mapeg® 200 DO, PPG), PEG-4 distearate (Kessco® 200 DS, Stepan), PEG-6 dilaurate (Kessco® PEG 300 DL, Stepan), PEG-6 dioleate (Kessco® PEG 300 DO, Stepan), PEG-6 distearate (Kessco® PEG 300 DS, Stepan), PEG-8 dilaurate (Mapeg® 400 DL, PPG), PEG-8 dioleate (Mapeg® 400 DO, PPG), PEG-8 distearate (Mapeg® 400 DS, PPG), PEG-10 dipalmitate (Polyaldo 2PKFG), PEG-12 dilaurate (Kessco® PEG 600 DL, Stepan), PEG-12 distearate (Kessco® PEG 600 DS, Stepan), PEG-12 dioleate (Mapeg® 600 DO, PPG), PEG-20 dilaurate (Kessco® PEG 1000 DL, Stepan), PEG-20 dioleate (Kessco® PEG 1000 DO, Stepan), PEG-20 distearate (Kessco® PEG 1000 DS, Stepan), PEG-32 dilaurate (Kessco® PEG 1540 DL, Stepan), PEG-32 dioleate (Kessco® PEG 1540 DO, Stepan), PEG-32 distearate (Kessco® PEG 1540 DS, Stepan), PEG-400 dioleate (Cithrol 4DO series, Croda), and PEG-400 distearate Cithrol 4DS series, Croda). Formulations of rifalazil according to the invention may include one or more of the polyethylene glycol fatty acid diesters above.

PEG-fatty acid mono- and di-ester mixtures may be used as excipients for the formulation of rifalazil. Examples of commercially available PEG-fatty acid mono- and di-ester mixtures include: PEG 4-150 mono, dilaurate (Kessco® PEG 200-6000 mono, Dilaurate, Stepan), PEG 4-150 mono, dioleate (Kessco® PEG 200-6000 mono, Dioleate, Stepan), and PEG 4-150 mono, distearate (Kessco® 200-6000 mono, Distearate, Stepan). Formulations of rifalazil according to the invention may include one or more of the PEG-fatty acid mono- and di-ester mixtures above.

In addition, polyethylene glycol glycerol fatty acid esters may be used as excipients for the formulation of rifalazil. Examples of commercially available polyethylene glycol glycerol fatty acid esters include: PEG-20 glyceryl laurate (Tagat® L, Goldschmidt), PEG-30 glyceryl laurate (Tagat® L2, Goldschmidt), PEG-15 glyceryl laurate (Glycerox L series, Croda), PEG-40 glyceryl laurate (Glycerox L series, Croda), PEG-20 glyceryl stearate (Capmul® EMG, ABITEC), and Aldo® MS-20 KFG, Lonza), PEG-20 glyceryl oleate (Tagat® O, Goldschmidt), and PEG-30 glyceryl oleate (Tagat® O2, Goldschmidt). Formulations of rifalazil according to the invention may include one or more of the polyethylene glycol and glycerol fatty acid esters above.

Alcohol-oil transesterification products may also be used as excipients for the formulation of rifalazil. Examples of commercially available alcohol-oil transesterification products include: PEG-3 castor oil (Nikkol CO-3, Nikko), PEG-5, 9, and 16 castor oil (ACCONON CA series, ABITEC), PEG-20 castor oil, (Emalex C-20, Nihon Emulsion), PEG-23 castor oil (Emulgante EL23), PEG-30 castor oil (Incrocas 30, Croda), PEG-35 castor oil (Incrocas-35, Croda), PEG-38 castor oil (Emulgante EL 65, Condea), PEG-40 castor oil (Emalex C-40, Nihon Emulsion), PEG-50 castor oil (Emalex C-50, Nihon Emulsion), PEG-56 castor oil (Eumulgin® PRT 56, Pulcra SA), PEG-60 castor oil (Nikkol CO-60TX, Nikko), PEG-100 castor oil, PEG-200 castor oil (Eumulgin® PRT 200, Pulcra SA), PEG-5 hydrogenated castor oil (Nikkol HCO-5, Nikko), PEG-7 hydrogenated castor oil (Cremophor WO7, BASF), PEG-10 hydrogenated castor oil (Nikkol HCO-10, Nikko), PEG-20 hydrogenated castor oil (Nikkol HCO-20, Nikko), PEG-25 hydrogenated castor oil (Simulsol® 1292, Seppic), PEG-30 hydrogenated castor oil (Nikkol HCO-30, Nikko), PEG-40 hydrogenated castor oil (Cremophor RH 40, BASF), PEG-45 hydrogenated castor oil (Cerex ELS 450, Auschem Spa), PEG-50 hydrogenated castor oil (Emalex HC-50, Nihon Emulsion), PEG-60 hydrogenated castor oil (Nikkol HCO-60, Nikko), PEG-80 hydrogenated castor oil (Nikkol HCO-80, Nikko), PEG-100 hydrogenated castor oil (Nikkol HCO-100, Nikko), PEG-6 corn oil (Labrafil® M 2125 CS, Gattefosse), PEG-6 almond oil (Labrafil® M 1966 CS, Gattefosse), PEG-6 apricot kernel oil (Labrafil® M 1944 CS, Gattefosse), PEG-6 olive oil (Labrafil® M 1980 CS, Gattefosse), PEG-6 peanut oil (Labrafil® M 1969 CS, Gattefosse), PEG-6 hydrogenated palm kernel oil (Labrafil® M 2130 BS, Gattefosse), PEG-6 palm kernel oil (Labrafil® M 2130 CS, Gattefosse), PEG-6 triolein (Labrafil® M 2735 CS, Gattefosse), PEG-8 corn oil (Labrafil® WL 2609 BS, Gattefosse), PEG-20 corn glycerides (Crovol M40, Croda), PEG-20 almond glycerides (Crovol A40, Croda), PEG-25 trioleate (TAGAT® TO, Goldschmidt), PEG-40 palm kernel oil (Crovol PK-70), PEG-60 corn glycerides (Crovol M70, Croda), PEG-60 almond glycerides (Crovol A70, Croda), PEG-4 caprylic/capric triglyceride (Labrafac® Hydro, Gattefosse), PEG-8 caprylic/capric glycerides (Labrasol, Gattefosse), PEG-6 caprylic/capric glycerides (SOFTIGEN®767, Huls), lauroyl macrogol-32 glyceride (GELUCIRE 44/14, Gattefosse), stearoyl macrogol glyceride (GELUCIRE 50/13, Gattefosse), mono, di, tri, tetra esters of vegetable oils and sorbitol (SorbitoGlyceride, Gattefosse), pentaerythrityl tetraisostearate (Crodamol PTIS, Croda), pentaerythrityl distearate (Albunol DS, Taiwan Surf.), pentaerythrityl tetraoleate (Liponate PO-4, Lipo Chem.), pentaerythrityl tetrastearate (Liponate PS-4, Lipo Chem.), pentaerythrityl tetracaprylate tetracaprate (Liponate PE-810, Lipo Chem.), and pentaerythrityl tetraoctanoate (Nikkol Pentarate 408, Nikko). Also included in this category of surfactants are esters of oil-soluble vitamins, such as vitamins A, D, E, K, etc. Thus, derivatives of these vitamins, such as tocopheryl PEG-1000 succinate (TPGS, available from Eastman), are also suitable surfactants. Formulations of rifalazil according to the invention may include one or more of the alcohol-oil transesterification products above.

Polyglycerized fatty acids may also be used as excipients for the formulation of rifalazil. Examples of commercially available polyglycerized fatty acids include: polyglyceryl-2 stearate (Nikkol DGMS, Nikko), polyglyceryl-2 oleate (Nikkol DGMO, Nikko), polyglyceryl-2 isostearate (Nikkol DGMIS, Nikko), polyglyceryl-3 oleate (Caprol® 3GO, ABITEC), polyglyceryl-4 oleate (Nikkol Tetraglyn 1-O, Nikko), polyglyceryl-4 stearate (Nikkol Tetraglyn 1-S, Nikko), polyglyceryl-6 oleate (Drewpol 6-1-O, Stepan), polyglyceryl-10 laurate (Nikkol Decaglyn 1-L, Nikko), polyglyceryl-10 oleate (Nikkol Decaglyn 1-O, Nikko), polyglyceryl-10 stearate (Nikkol Decaglyn 1-S, Nikko), polyglyceryl-6 ricinoleate (Nikkol Hexaglyn PR-15, Nikko), polyglyceryl-10 linoleate (Nikkol Decaglyn 1-LN, Nikko), polyglyceryl-6 pentaoleate (Nikkol Hexaglyn 5-O, Nikko), polyglyceryl-3 dioleate (Cremophor GO32, BASF), polyglyceryl-3 distearate (Cremophor GS32, BASF), polyglyceryl-4 pentaoleate (Nikkol Tetraglyn 5-O, Nikko), polyglyceryl-6 dioleate (Caprol® 6G20, ABITEC), polyglyceryl-2 dioleate (Nikkol DGDO, Nikko), polyglyceryl-10 trioleate (Nikkol Decaglyn 3-O, Nikko), polyglyceryl-10 pentaoleate (Nikkol Decaglyn 5-O, Nikko), polyglyceryl-10 septaoleate (Nikkol Decaglyn 7-O, Nikko), polyglyceryl-10 tetraoleate (Caprol® 10G4O, ABITEC), polyglyceryl-10 decaisostearate (Nikkol Decaglyn 10-IS, Nikko), polyglyceryl-101 decaoleate (Drewpol 10-10-O, Stepan), polyglyceryl-10 mono, dioleate (Caprol® PGE 860, ABITEC), and polyglyceryl polyricinoleate (Polymuls, Henkel). Formulations of rifalazil according to the invention may include one or more of the polyglycerized fatty acids above.

In addition, propylene glycol fatty acid esters may be used as surfactants for the formulation of rifalazil. Examples of commercially available propylene glycol fatty acid esters include: propylene glycol monocaprylate (Capryol 90, Gattefosse), propylene glycol monolaurate (Lauroglycol 90, Gattefosse), propylene glycol oleate (Lutrol OP2000, BASF), propylene glycol myristate (Mirpyl), propylene glycol monostearate (LIPO PGMS, Lipo Chem.), propylene glycol hydroxystearate, propylene glycol ricinoleate (PROPYMULS, Henkel), propylene glycol isostearate, propylene glycol monooleate (Myverol P-O6, Eastman), propylene glycol dicaprylate dicaprate (Captex® 200, ABITEC), propylene glycol dioctanoate (Captex® 800, ABITEC), propylene glycol caprylate caprate (LABRAFAC PG, Gattefosse), propylene glycol dilaurate, propylene glycol distearate (Kessco® PGDS, Stepan), propylene glycol dicaprylate (Nikkol Sefsol 228, Nikko), and propylene glycol dicaprate (Nikkol PDD, Nikko). Formulations of rifalazil according to the invention may include one or more of the propylene glycol fatty acid esters above.

Mixtures of propylene glycol esters and glycerol esters may also be used as lipophilic surfactants for the formulation of rifalazil. One preferred mixture is composed of the oleic acid esters of propylene glycol and glycerol (Arlacel 186). Examples of these surfactants include: oleic (ATMOS 300, ARLACEL 186, ICI), and stearic (ATMOS 150). Formulations of rifalazil according to the invention may include one or more of the mixtures of propylene glycol esters and glycerol esters above.

Furthermore, mono- and diglycerides may be used as lipophilic surfactants for the formulation of rifalazil. Examples of commercially available mono- and diglycerides include: monopalmitolein (C16:1) (Larodan), monoelaidin (C18:1) (Larodan), monocaproin (C6) (Larodan), monocaprylin (Larodan), monocaprin (Larodan), monolaurin (Larodan), glyceryl monomyristate (C14) (Nikkol MGM, Nikko), glyceryl monooleate (C18:1) (PECEOL, Gattefosse), glyceryl monooleate (Myverol, Eastman), glycerol monooleate/linoleate (OLICINE, Gattefosse), glycerol monolinoleate (Maisine, Gattefosse), glyceryl ricinoleate (Softigen® 701, Huls), glyceryl monolaurate (ALDO® MLD, Lonza), glycerol monopalmitate (Emalex GMS-P, Nihon), glycerol monostearate (Capmul® GMS, ABITEC), glyceryl mono- and dioleate (Capmul® GMO-K, ABITEC), glyceryl palmitic/stearic (CUTINA MD-A, ESTAGEL-G18), glyceryl acetate (Lamegin® EE, Grunau GmbH), glyceryl laurate (Imwitor® 312, Huls), glyceryl citrate/lactate/oleate/linoleate (Imwitor® 375, Huls), glyceryl caprylate (Imwitor® 308, Huls), glyceryl caprylate/caprate (Capmul® MCM, ABITEC), caprylic acid mono- and diglycerides (Imwitor® 988, Huls), caprylic/capric glycerides (Imwitor® 742, Huls), Mono- and diacetylated monoglycerides (Myvacet® 9-45, Eastman), glyceryl monostearate (Aldo® MS, Arlacel 129, ICI), lactic acid esters of mono and diglycerides (LAMEGIN GLP, Henkel), dicaproin (C6) (Larodan), dicaprin (C10) (Larodan), dioctanoin (C8) (Larodan), dimyristin (C14) (Larodan), dipalmitin (C16) (Larodan), distearin (Larodan), glyceryl dilaurate (C12) (Capmul® GDL, ABITEC), glyceryl dioleate (Capmul® GDO, ABITEC), glycerol esters of fatty acids (GELUCIRE 39/01, Gattefosse), dipalmitolein (C16:1) (Larodan), 1,2 and 1,3-diolein (C18:1) (Larodan), dielaidin (C18:1) (Larodan), and dilinolein (C18:2) (Larodan). Formulations of rifalazil according to the invention may include one or more of the mono- and diglycerides above.

Sterol and sterol derivatives may also be used as excipients for the formulation of rifalazil. Examples of commercially available sterol and sterol derivatives include: cholesterol, sitosterol, lanosterol, PEG-24 cholesterol ether (Solulan C-24, Amerchol), PEG-30 cholestanol (Phytosterol GENEROL series, Henkel), PEG-25 phytosterol (Nikkol BPSH-25, Nikko), PEG-5 soyasterol (Nikkol BPS-5, Nikko), PEG-10 soyasterol (Nikkol BPS-10, Nikko), PEG-20 soyasterol (Nikkol BPS-20, Nikko), and PEG-30 soyasterol (Nikkol BPS-30, Nikko). Formulations of rifalazil according to the invention may include one or more of the sterol and sterol derivatives above.

Polyethylene glycol sorbitan fatty acid esters may also be used as surfactants for the formulation of rifalazil. Examples of commercially available polyethylene glycol sorbitan fatty acid esters include: PEG-10 sorbitan laurate (Liposorb L-10, Lipo Chem.), PEG-20 sorbitan monolaurate (Tween® 20, Atlas/ICI), PEG-4 sorbitan monolaurate (Tween® 21, Atlas/ICI), PEG-80 sorbitan monolaurate (Hodag PSML-80, Calgene), PEG-6 sorbitan monolaurate (Nikkol GL-1, Nikko), PEG-20 sorbitan monopalmitate (Tween® 40, Atlas/ICI), PEG-20 sorbitan monostearate (Tween® 60, Atlas/ICI), PEG-4 sorbitan monostearate (Tween® 61, Atlas/ICI), PEG-8 sorbitan monostearate (DACOL MSS, Condea), PEG-6 sorbitan monostearate (Nikkol TS106, Nikko), PEG-20 sorbitan tristearate (Tween® 65, Atlas/ICI), PEG-6 sorbitan tetrastearate (Nikkol GS-6, Nikko), PEG-60 sorbitan tetrastearate (Nikkol GS-460, Nikko), PEG-5 sorbitan monooleate (Tween® 81, Atlas/ICI), PEG-6 sorbitan monooleate (Nikkol TO-106, Nikko), PEG-20 sorbitan monooleate (Tween® 80, Atlas/ICI), PEG-40 sorbitan oleate (Emalex ET 8040, Nihon Emulsion), PEG-20 sorbitan trioleate (Tween® 85, Atlas/ICI), PEG-6 sorbitan tetraoleate (Nikkol GO-4, Nikko), PEG-30 sorbitan tetraoleate (Nikkol GO-430, Nikko), PEG-40 sorbitan tetraoleate (Nikkol GO-440, Nikko), PEG-20 sorbitan monoisostearate (Tween® 120, Atlas/ICI), PEG sorbitol hexaoleate (Atlas G-1086, ICI), polysorbate 80 (Tween® 80, Pharma), polysorbate 85 (Tween® 85, Pharma), polysorbate 20 (Tween® 20, Pharma), polysorbate 40 (Tween® 40, Pharma), polysorbate 60 (Tween® 60, Pharma), and PEG-6 sorbitol hexastearate (Nikkol GS-6, Nikko). Formulations of rifalazil according to the invention may include one or more of the polyethylene glycol sorbitan fatty acid esters above.

In addition, polyethylene glycol alkyl ethers may be used as surfactants for the formulation of rifalazil. Examples of commercially available polyethylene glycol alkyl ethers include: PEG-2 oleyl ether, oleth-2 (Brij 92/93, Atlas/ICI), PEG-3 oleyl ether, oleth-3 (Volpo 3, Croda), PEG-5 oleyl ether, oleth-5 (Volpo 5, Croda), PEG-10 oleyl ether, oleth-10 (Volpo 10, Croda), PEG-20 oleyl ether, oleth-20 (Volpo 20, Croda), PEG-4 lauryl ether, laureth-4 (Brij 30, Atlas/ICI), PEG-9 lauryl ether, PEG-23 lauryl ether, laureth-23 (Brij 35, Atlas/ICI), PEG-2 cetyl ether (Brij 52, ICI), PEG-10 cetyl ether (Brij 56, ICI), PEG-20 cetyl ether (BriJ 58, ICI), PEG-2 stearyl ether (Brij 72, ICI), PEG-10 stearyl ether (Brij 76, ICI), PEG-20 stearyl ether (Brij 78, ICI), and PEG-100 stearyl ether (Brij 700, ICI). Formulations of rifalazil according to the invention may include one or more of the polyethylene glycol alkyl ethers above.

Sugar esters may also be used as surfactants for the formulation of rifalazil. Examples of commercially available sugar esters include: sucrose distearate (SUCRO ESTER 7, Gattefosse), sucrose distearate/monostearate (SUCRO ESTER 11, Gattefosse), sucrose dipalmitate, sucrose monostearate (Crodesta F-160, Croda), sucrose monopalmitate (SUCRO ESTER 15, Gattefosse), and sucrose monolaurate (Saccharose monolaurate 1695, Mitsubisbi-Kasei). Formulations of rifalazil according to the invention may include one or more of the sugar esters above.

Polyethylene glycol alkyl phenols are also useful as surfactants for the formulation of rifalazil. Examples of commercially available polyethylene glycol alkyl phenols include: PEG-10-100 nonylphenol series (Triton X series, Rohm & Haas) and PEG-15-100 octylphenol ether series (Triton N-series, Rohm & Haas). Formulations of rifalazil according to the invention may include one or more of the polyethylene glycol alkyl phenols above.

Sorbitan fatty acid esters may also be used as surfactants for the formulation of rifalazil. Examples of commercially sorbitan fatty acid esters include: sorbitan monolaurate (Span-20, Atlas/ICI), sorbitan monopalmitate (Span-40, Atlas/ICI), sorbitan monooleate (Span-80, Atlas/ICI), sorbitan monostearate (Span-60, Atlas/ICI), sorbitan trioleate (Span-85, Atlas/ICI), sorbitan sesquioleate (Arlacel-C, ICI), sorbitan tristearate (Span-65, Atlas/ICI), sorbitan monoisostearate (Crill 6, Croda), and sorbitan sesquistearate (Nikkol SS-15, Nikko). Formulations of rifalazil according to the invention may include one or more of the sorbitan fatty acid esters above.

Esters of lower alcohols (C₂ to C₄) and fatty acids (C₈ to C₁₈) are suitable lipophilic surfactants for use in the invention. Examples of these surfactants include: ethyl oleate (Crodamol EO, Croda), isopropyl myristate (Crodamol IPM, Croda), isopropyl palmitate (Crodamol IPP, Croda), ethyl linoleate (Nikkol VF-E, Nikko), and isopropyl linoleate (Nikkol VF-IP, Nikko). Formulations of rifalazil according to the invention may include one or more of the lower alcohol fatty acid esters above.

In addition, ionic surfactants may be used as excipients for the formulation of rifalazil. Examples of useful ionic surfactants include: sodium caproate, sodium caprylate, sodium caprate, sodium laurate, sodium myristate, sodium myristolate, sodium palmitate, sodium palmitoleate, sodium oleate, sodium ricinoleate, sodium linoleate, sodium linolenate, sodium stearate, sodium lauryl sulfate (dodecyl), sodium tetradecyl sulfate, sodium lauryl sarcosinate, sodium dioctyl sulfosuccinate, sodium cholate, sodium taurocholate, sodium glycocholate, sodium deoxycholate, sodium taurodeoxycholate, sodium glycodeoxycholate, sodium ursodeoxycholate, sodium chenodeoxycholate, sodium taurochenodeoxycholate, sodium glyco cheno deoxycholate, sodium cholylsarcosinate, sodium N-methyl taurocholate, egg yolk phosphatides, hydrogenated soy lecithin, dimyristoyl lecithin, lecithin, hydroxylated lecithin, lysophosphatidylcholine, cardiolipin, sphingomyelin, phosphatidylcholine, phosphatidyl ethanolamine, phosphatidic acid, phosphatidyl glycerol, phosphatidyl serine, diethanolamine, phospholipids, polyoxyethylene-10 oleyl ether phosphate, esterification products of fatty alcohols or fatty alcohol ethoxylates, with phosphoric acid or anhydride, ether carboxylates (by oxidation of terminal OH group of, fatty alcohol ethoxylates), succinylated monoglycerides, sodium stearyl fumarate, stearoyl propylene glycol hydrogen succinate, mono/diacetylated tartaric acid esters of mono- and diglycerides, citric acid esters of mono-, diglycerides, glyceryl-lacto esters of fatty acids, acyl lactylates, lactylic esters of fatty acids, sodium stearoyl-2-lactylate, sodium stearoyl lactylate, alginate salts, propylene glycol alginate, ethoxylated alkyl sulfates, alkyl benzene sulfones, α-olefin sulfonates, acyl isethionates, acyl taurates, alkyl glyceryl ether sulfonates, sodium octyl sulfosuccinate, sodium undecylenamideo-MEA-sulfosuccinate, hexadecyl triammonium bromide, decyl trimethyl ammonium bromide, cetyl trimethyl ammonium bromide, dodecyl ammonium chloride, alkyl benzyldimethylammonium salts, diisobutyl phenoxyethoxydimethyl benzylammonium salts, alkylpyridinium salts, betaines (trialkylglycine), lauryl betaine (N-lauryl,N,N-dimethylglycine), and ethoxylated amines (polyoxyethylene-15 coconut amine). For simplicity, typical counterions are provided above. It will be appreciated by one skilled in the art, however, that any bioacceptable counterion may be used. For example, although the fatty acids are shown as sodium salts, other cation counterions can also be used, such as, for example, alkali metal cations or ammonium. Formulations of rifalazil according to the invention may include one or more of the ionic surfactants above.

Many of the foregoing surfactants are micelle-forming in aqueous and intestinal media. However, with the compositions of the present invention non-micellar aggregates, such as emulsions and microemulsions, can also be formed in aqueous and intestinal media. The formation of micelles can be monitored using any of several standard techniques known in the art, including surface tension measurements, solubilization of water insoluble dye, conductivity measurements, and light scattering, among others. In all of these methods, an abrupt change in some physicochemical property is measured as a function of surfactant concentration. The abrupt change occurs when the concentration of surfactant is sufficient to form micelles. Above this concentration, also known as the critical micelle concentration (CMC), micelles are present in solution. Above the CMC, the concentration of micelles increases whereas the concentration of monomeric surfactant in equilibrium with micelles remains constant.

Various MW sizes of polyoxyethylene glycols (PEG) are suitable hydrophilic co-solvents for use in the invention. Polyoxyethylene glycol polymers which can be used in the methods and compositions of the invention can be from 200 Da to 10,000 Da, more preferably from 200 Da to 2,000 Da, in size. Specific examples include PEG-200, PEG-300, PEG-400, PEG-600, PEG-800, PEG-1,000, PEG-1,200, PEG-1,500, PEG 2000 and combinations thereof.

Methods for making formulations for oral administration are found, for example, in “Remington: The Science and Practice of Pharmacy” (20th ed., ed. A. R. Gennaro, 2000, Lippincott Williams & Wilkins). Formulations for oral administration (e.g., tablets, pills, caplets, hard capsules, and soft capsules) may, for example, contain any one or combination of the excipients described above along with other excipients as needed. Liquid-filled capsules can include any of the excipients described herein. The capsule will contain from, for example, 0.1 to about 100 mg of rifalazil. Liquid-filled capsules may, for example, contain either solutions or suspensions of rifalazil, depending upon the concentration of rifalazil within the capsule and the excipients used in the formulation.

The filled formulation can also be a semi-solid formulation, e.g., solid at ambient temperature but liquid at physiological temperature. Semi-solid formulations can be made, for example, by including a sufficient amount of high molecular weight PEG (i.e., greater that 600 Da, preferably 1,500 Da) in the formulation. Alternatively, inclusion of a surfactant having a melting point above 37° C. can result in a semi-solid formulation. Formulations of M4 and M5 (see Table 9) are examples semi-solid formulations.

Rifalazil may be formulated as a pharmaceutically acceptable salt, such as a non-toxic acid addition salt or metal complexe that are commonly used in the pharmaceutical industry. Examples of acid addition salts include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids or the like; polymeric acids such as tannic acid, carboxymethyl cellulose, or the like; and inorganic acid such as hydrochloric acid, hydrobromic acid, sulfuric acid, phosphoric acid, or the like. Metal complexes include zinc, iron, and the like.

Many strategies can be pursued to obtain sustained or controlled release in which the rate of release outweighs the rate of metabolism of the therapeutic compound. For example, sustained or controlled release can be obtained by the appropriate selection of formulation parameters and ingredients, including, e.g., single or multiple unit capsule compositions, by varying the amount of hydrophilic polymer present in a liquid-filled rifalazil capsule of the invention, or by varying the amount of gelling agent in the formulated capsule or by using a surfactant that is semi-solid at ambient temperature. Other controlled released polymeric excipients can also be used in the compositions of the present invention.

Other Therapeutic Agents

The rifalazil formulations described herein may also include a second therapeutic agent including, for example, another antibiotic, an anesthetic, an antimicrobial agent, a zinc salt, or an anti-inflammatory agent (e.g., an non-steroidal anti-inflammatory or a steroid).

Antibiotics that can be admixed with the pharmaceutical compositions of the invention include: aminoglycosides, such as amikacin, apramycin, arbekacin, bambermycins, butirosin, dibekacin, dihydrostreptomycin, fortimicin(s), fradiomycin, gentamicin, ispamicin, kanamycin, micronomicin, neomycin, neomycin undecylenate, netilmicin, paromomycin, ribostamycin, sisomicin, spectinomycin, streptomycin, streptonicozid, and tobramycin; amphenicols, such as azidamfenicol, chloramphenicol, chloramphenicol palmirate, chloramphenicol pantothenate, florfenicol, and thiamphenicol; ansamycins, such as rifampin, rifabutin, rifapentine, and rifaximin; β-Lactams, such as amidinocillin, amdinocillin, pivoxil, amoxicillin, ampicillin, aspoxicillin, azidocillin, azlocillin, bacampicillin, benzylpenicillinic acid, benzylpenicillin, carbenicillin, carfecillin, carindacillin, clometocillin, cloxacillin, cyclacillin, dicloxacillin, diphenicillin, epicillin, fenbenicillin, floxicillin, hetacillin, lenampicillin, metampicillin, methicillin, mezlocillin, nafcillin, oxacillin, penamecillin, penethamate hydriodide, penicillin G benethamine, penicillin G benzathine, penicillin G benzhydrylamine, penicillin G calcium, penicillin G hydragamine, penicillin G potassium, penicillin G, procaine, penicillin N, penicillin O, penicillin V, penicillin V benzathine, penicillin V hydrabamine, penimepicycline, phenethicillin, piperacillin, pivapicillin, propicillin, quinacillin, sulbenicillin, talampicillin, temocillin and ticarcillin; carbapenems, such as imipenem; cephalosporins, such as 1-carba (dethia) cephalosporin, cefactor, cefadroxil, cefamandole, cefatrizine, cefazedone, cefazolin, cefixime, cefmenoxime, cefodizime, cefonicid, cefoperazone, ceforanide, cefotaxime, cefotiam, cefpimizole, cefpirimide, cefpodoxime proxetil, cefroxadine, cefsulodin, ceftazidime, cefteram, ceftezole, ceftibuten, ceftizoxime, ceftriaxone, cefuroxime, cef*uizonam, cephacetrile sodium, cephalexin, cephaloglycin, cephaloridine, cephalosporin, cephalothin, cephapirin sodium, cephradine, pivcefalexin, cephalothin, cefaclor, cefotetan, cefprozil, loracarbef, cefetamet, and cefepime; cephamycins such as cefbuperazone, cefmetazole, cefminox, cefetan, and cefoxitin; monobactams such as aztreonam, carumonam, and tigemonan; oxacephems such as flomoxef and moxolactam; lincosamides such as clindamycin and lincomycin; macrolides such as azithromycin, carbomycin, clarithromycin, erythromycin(s) and derivatives, josamycin, leucomycins, midecamycins, miokamycin, oleandomycin, primycin, rokitamycin, rosaramicin, roxithromycin, spiramycin and troleandomycin; polypeptides such as amphomycin, bacitracin, capreomycin, colistin, enduracidin, enylomycin, fusafungine, gramicidin(s), gramicidin S, mikamycin, polymyxin, polymyxin .β-methanesulfonic acid, pristinamycin, ristocetin, teicoplanin, thiostrepton, tuberactinomycin, tyrocidine, tyrothricin, vancomycin, viomycin(s), virginiamycin and zinc bacitracin; tetracyclines such as spicycline, chlortetracycline, clomocycline, demeclocycline, doxycycline, guamecycline, lymecycline, meclocycline, methacycline, minocycline, oxytetracycline, penimepicycline, pipacycline, rolitetracycline, sancycline, senociclin and tetracycline; and 2,4-diaminopyrimidines such as brodimoprim, tetroxoprim and trimethoprim; nitrofurans such as furaltadone, furazolium, nifuradene, nifuratel, nifurfoline, nifurpirinol, nifurprazine, nifurtoinol and nitrofurantoin; quinolones such as amifloxacin, cinoxacin, ciprofloxacin, difloxacin, enoxacin, fleroxacin, flumequine, lomefloxacin, miloxacin, nalidixic acid, norfloxacin, ofloxacin, oxolinic acid, perfloxacin, pipemidic acid, piromidic acid, rosoxacin, temafloxacin, and tosufloxacin; sulfonamides such as acetyl sulfamethoxypyrazine, acetyl sulfisoxazole, azosulfamide, benzylsulfamide, chloramine-β, chloramine-T, dichloramine-T, formosulfathiazole, N₂-formyl-sulfisomidine, N₄-β-D-glucosylsulfanilamide, mafenide, 4′-(methyl-sulfamoyl)sulfanilanilide, p-nitrosulfathiazole, noprylsulfamide, phthalylsulfacetamide, phthalylsulfathiazole, salazosulfadimidine, succinylsulfathiazole, sulfabenzamide, sulfacetamide, sulfachlorpyridazine, sulfachrysoidine, sulfacytine, sulfadiazine, sulfadicramide, sulfadimethoxine, sulfadoxine, sulfaethidole, sulfaguanidine, sulfaguanol, sulfalene, sulfaloxic acid, sulfamerazine, sulfameter, sulfamethazine, sulfamethizole, sulfamethomidine, sulfamethoxazole, sulfamethoxypyridazine, sulfametrole, sulfamidochrysoidine, sulfamoxole, sulfanilamide, sulfanilamidomethanesulfonic acid triethanolamine salt, 4-sulfanilamidosalicyclic acid, N₄-sulfanilylsulfanilamide, sulfanilylurea, N-sulfanilyl-3,4-xylamide, sulfanitran, sulfaperine, sulfaphenazole, sulfaproxyline, sulfapyrazine, sulfapyridine, sulfasomizole, sulfasymazine, sulfathiazole, sulfathiourea, sulfatolamide, sulfisomidine and sulfisoxazole; sulfones, such as acedapsone, acediasulfone, acetosulfone, dapsone, diathymosulfone, glucosulfone, solasulfone, succisulfone, sulfanilic acid, p-sulfanilylbenzylamine, p,p′-sulfonyldianiline-N,N′ digalactoside, sulfoxone and thiazolsulfone; lipopeptides such as daptomycin; oxazolidones such as linezolid; ketolides such as telithromycin; and miscellaneous antibiotics such as clofoctol, hexedine, magainins, methenamine, methenamine anhydromethylene-citrate, methenamine hippurate, methenamine mandelate, methenamine sulfosalicylate, nitroxoline, squalamine, xibornol, cycloserine, mupirocin, and tuberin.

Preferred non-steroidal anti-inflammatory agents include, for example, detoprofen, diclofenac, diflunisal, etodolac, fenoprofen, flurbiprofen, indomethacin, ketoprofen, mechlofenameate, mefenamic acid, meloxicam, nabumeone, naproxen sodium, oxaprozin, piroxicam, sulindac, tolmeting, celecoxib, rofecoxib, choline salicylate, salsate, sodium salicylate, magnesium salicylate, aspirin, ibuprofen, paracetamol, acetaminophen, and pseudoephedrine, and preferred steroids include, for example, hydrocortisone, prednisone, fluprednisolone, triamcinolone, dexamethasone, betamethasone, cortisone, prednilosone, methylprednisolone, fluocinolone acetonide, flurandrenolone acetonide, and fluorometholone.

Preferred anesthetics include, for example, benzocaine, butamben picrate, tetracaine, dibucaine, prilocaine, etidocaine, mepivacaine, bupivicaine, and lidocaine.

Preferred zinc salts include, for example, zinc sulfate, zinc chloride, zinc acetate, zinc phenol sulfonate, zinc borate, zinc bromide, zinc nitrate, zinc glycerophosphate, zinc benzoate, zinc carbonate, zinc citrate, zinc hexafluorosilicate, zinc diacetate trihydrate, zinc oxide, zinc peroxide, zinc salicylate, zinc silicate, zinc stannate, zinc tannate, zinc titanate, zinc tetrafluoroborate, zinc gluconate, and zinc glycinate.

All of the therapeutic agents employed in the pharmaceutical compositions of the invention can be used in the dose ranges currently known and used for these agents. Different concentrations may be employed depending on the clinical condition of the patient, the goal of therapy (treatment or prophylaxis), the anticipated duration, and the severity of the infection or disease for which a pharmaceutical composition of the invention is being administered. Additional considerations in dose selection include the type of infection, age of the patient (e.g., pediatric, adult, or geriatric), general health, and comorbidity. Determining what concentrations to employ are within the skills of the pharmacist, medicinal chemist, or medical practitioner formulating pharmaceutical composition of the invention in combination with other therapeutic agents.

Therapy

The pharmaceutical compositions described herein can be used to treat or prevent bacterial infections as well as diseases associated with bacterial infections.

Diseases associated with bacterial infections include, but are not limited to, multiple sclerosis (MS), rheumatoid arthritis (RA), inflammatory bowel disease (IBD), interstitial cystitis (IC), fibromyalgia (FM), autonomic nervous dysfunction (AND, neural-mediated hypotension); pyoderma gangrenosum (PG), chronic fatigue (CF), and chronic fatigue syndrome (CFS).

Several lines of evidence have led to the establishment of a link between bacterial infections and a broad set of inflammatory, autoimmune, and immune deficiency diseases. Thus, the present invention describes methods for treating chronic diseases associated with a persistent infection, such as autoimmune diseases, inflammatory diseases and diseases that occur in immuno-compromised individuals by treating the non-multiplying form of the infection in an individual in need thereof, by administering a rifalazil formulation described herein, or such a rifalazil formulation in conjunction with an antibiotic effective against multiplying bacteria. Progress of the treatment can be evaluated, using the diagnostic tests known in the art, to determine the presence or absence of the bacteria. Physical improvement in the conditions and symptoms typically associated with the disease to be treated can also be evaluated. Based upon these evaluating factors, the physician can maintain or modify the anti-bacterial therapy accordingly.

The therapies described herein can be used for the treatment of chronic immune and autoimmune diseases when patients are demonstrated to have a bacterial infection. These diseases include, but are not limited to, chronic hepatitis, systemic lupus erythematosus, arthritis, thyroidosis, scleroderma, diabetes mellitus, Graves' disease, Beschet's disease, and graft versus host disease (graft rejection). The therapies of this invention can also be used to treat any disorders in which a bacterial infection is a factor or co-factor.

Thus, the present invention can be used to treat a range of disorders in addition to the above immune and autoimmune diseases when demonstrated to be associated with chlamydial infection by the methods of detection described herein; for example, various infections, many of which produce inflammation as primary or secondary symptoms, including, but not limited to, sepsis syndrome, cachexia, circulatory collapse and shock resulting from acute or chronic bacterial infection, acute and chronic parasitic and/or infectious diseases from bacterial, viral or fungal sources, such as a HIV, AIDS (including symptoms of cachexia, autoimmune disorders, AIDS dementia complex and infections) can be treated.

Among the various inflammatory diseases, there are certain features that are generally agreed to be characteristic of the inflammatory process. These include fenestration of the microvasculature, leakage of the elements of blood into the interstitial spaces, and migration of leukocytes into the inflamed tissue. On a macroscopic level, this is usually accompanied by the familiar clinical signs of erythema, edema, tenderness (hyperalgesia), and pain. Inflammatory diseases, such as chronic inflammatory pathologies and vascular inflammatory pathologies, including chronic inflammatory pathologies such as aneurysms, hemorrhoids, sarcoidosis, chronic inflammatory bowel disease, ulcerative colitis, and Crohn's disease and vascular inflammatory pathologies, such as, but not limited to, disseminated intravascular coagulation, atherosclerosis, and Kawasaki's pathology are also suitable for treatment by methods described herein. The invention can also be used to treat inflammatory diseases such as coronary artery disease, hypertension, stroke, asthma, chronic hepatitis, multiple sclerosis, peripheral neuropathy, chronic or recurrent sore throat, laryngitis, tracheobronchitis, chronic vascular headaches (including migraines, cluster headaches and tension headaches) and pneumonia when demonstrated to be pathogenically related to a bacterial infection.

Treatable disorders when associated with a bacterial infection also include, but are not limited to, neurodegenerative diseases, including, but not limited to, demyelinating diseases, such as multiple sclerosis and acute transverse myelitis; extrapyramidal and cerebellar disorders, such as lesions of the corticospinal system; disorders of the basal ganglia or cerebellar disorders; hyperkinetic movement disorders such as Huntington's Chorea and senile chorea; drug-induced movement disorders, such as those induced by drugs which block CNS dopamine receptors; hypokinetic movement disorders, such as Parkinson's disease; progressive supranucleo palsy; cerebellar and spinocerebellar disorders, such as astructural lesions of the cerebellum; spinocerebellar degenerations (spinal ataxia, Friedreich's ataxia, cerebellar cortical degenerations, multiple systems degenerations (Mencel, Dejerine-Thomas, Shi-Drager, and Machado-Joseph)); and systemic disorders (Refsum's disease, abetalipoprotemia, ataxia, telangiectasia, and mitochondrial multi-system disorder); demyelinating core disorders, such as multiple sclerosis, acute transverse myelitis; disorders of the motor unit, such as neurogenic muscular atrophies (anterior horn cell degeneration, such as amyotrophic lateral sclerosis, infantile spinal muscular atrophy and juvenile spinal muscular atrophy); Alzheimer's disease; Down's Syndrome in middle age; Diffuse Lewy body disease; senile dementia of Lewy body type; Wernicke-Korsakoff syndrome; chronic alcoholism; Creutzfeldt-Jakob disease; subacute sclerosing panencephalitis, Hallerrorden-Spatz disease; and dementia pugilistica.

It is also recognized that malignant pathologies involving tumors or other malignancies, such as, but not limited to leukemias (acute, chronic myelocytic, chronic lymphocytic and/or myelodyspastic syndrome); lymphomas (Hodgkin's and non-Hodgkin's lymphomas, such as malignant lymphomas (Burkitt's lymphoma or mycosis fungoides)); carcinomas (such as colon carcinoma) and metastases thereof; cancer-related angiogenesis; infantile hemangiomas; and alcohol-induced hepatitis. Ocular neovascularization, psoriasis, duodenal ulcers, angiogenesis of the female reproductive tract, can also be treated when demonstrated by the diagnostic procedures described herein to be associated with a bacterial infection.

Peripheral Artery Disease

Atherosclerosis and its complications lead to half of all adult deaths in the United States and other western societies, and its incidence is increasing in developing countries. Evidence suggesting that atherosclerosis is a chronic inflammatory disease has led to considerable research into the role played by infectious agents. Although a range of viruses and bacteria have been implicated in atherosclerosis, Chlamydia (C.) pneumoniae shows the strongest association to date in a range of epidemiological and experiment-based studies.

Peripheral arterial occlusive disease (PAOD; also referred to as peripheral arterial disease (PAD)) results either from atherosclerotic or inflammatory processes producing arterial stenosis, or from thrombus formation associated with underlying atherosclerotic disease. A common site for PAOD is in the lower limbs. This process of atherosclerosis causes intimal thickening and plaque formation encroaching the arterial lumen, decreasing the effective luminal radius of afflicted arterial segments, producing an anatomic and sometimes functional obstruction to blood flow. When these conditions arise, an increase in vascular resistance can lead to a reduction in distal perfusion pressure and blood flow. PAOD affects 20% to 30% of men and women age 50 years and older seen in general medical practices, and is associated with other forms of coronary artery disease, specifically atherosclerosis and general functional impairments (e.g., slower walking ability or decreased endurance) and may have a significant negative impact on the quality of independent living. PAOD can be reliably detected with doppler-recorded systolic pressures as a differential in the ankle-brachial ratio of these pressures.

The invention provides a method for treating an atherosclerosis-associated disease, such as atherosclerosis or peripheral artery disease by administering (i) rifalazil and (ii) a lipophilic antioxidant to a patient with the disease in an amount, that together, is effective to treat the disease. Using the methods of the invention, the two components are administered within 14 days of each other, or simultaneously. The two components may be formulated together as a single composition, or may be formulated and administered separately. In the combination therapies of the invention, the dosage and frequency of administration of each component of the combination can be controlled independently. For example, the lipophilic antioxidant may be administered three times per day, while the rifalazil may be administered once per week. Combination therapy may be given in on-and-off cycles that include rest periods so that the patient's body has a chance to recover from any as yet unforeseen side effects. Ultimately, the prescribers will decide the appropriate amount and dosage regimen. Additionally, an effective amount may can be that amount of compound in the combination of the invention that is safe and efficacious in the treatment of a patient having an atherosclerosis-associated disease over each component used alone as determined and approved by a regulatory authority (such as the U.S. Food and Drug Administration).

The combination of rifalazil and a lipophilic antioxidant can be administered, for example, to reduce C. pneumoniae burden and plaque area stenosis in atherosclerotic patients, especially where C. pneumoniae infection has exacerbated plaque deposition. The administration of a pharmaceutical composition of the invention invention can be used to accomplish any of the following: (i) reducing the occurrence and/or severity of intermittent claudication; (ii) reducing the functional impairments associated with the progression of PAOD; (iii) reducing the number and/or frequency of vascular interventions over time and related clinical complications over time; (iv) reducing the number and/or frequency of cardiovascular complications over time; (v) reducing localized inflammation in an atherosclerotic plaque; (vi) reducing the size of an atherosclerotic plaque; (vii) reducing the level of one or more inflammatory biomarkers (e.g., C-reactive protein, IL-6, IL-11, lipoprotein-associated phospholipase A2, fractalkine, monocyte chemotactic protein 1, neopterin, tumor necrosis factor receptors I and II, selectin, fibrinogen, ICAM-1, VCAM-1, myeloperoxidase); (viii) reducing the clinical complications associated with angioplasty and/or stent placement; (ix) reducing intimal hyperplasia and in-stent and peri-stent restenosis that occur after stent placement; (x) reducing vascular smooth muscle cell proliferation and/or the cellular and molecular products of vascular smooth muscle cell proliferation (including those mediated by the Toll-Like Receptor-2 pathways (see Yang et al. Arterioscler Thromb Vasc Biol., 25: 2308-2314, 2005)); or (xi) restoring endothelial function and capability in a patient.

The following examples are put forth so as to provide those of ordinary skill in the art with a complete disclosure and description of how the methods and compounds claimed herein are performed, made, and evaluated, and are intended to be purely exemplary of the invention and are not intended to be limiting.

### EXAMPLE 1

### Preparation of Liquid-Filled Capsules Containing 2.5 mg of Rifalazil

PEG-35 castor oil (Cremophor ELP), ascorbylpalmitate, Pluronic® F68, PEG 400, water, BHT, and rifalazil were mixed in proportions as provided below in Table 2. Capsules were filled with the liquid to produce liquid-filled capsules containing 2.5 mg of rifalazil each. The total fill weight per capsule was calculated based on target fill volume of 0.6 mL and density of 1.0421 g/mL.

TABLE 2
(Formulation A)

| Excipient | Amount (mg) per capsule | % (w/w) |
|---|---|---|
| Rifalazil (ABI-1648) | 2.5 | 0.40 |
| Ascorbyl Palmitate | 12.46 | 1.99 |
| Cremophor ELP | 517.08 | 82.70 |
| Pluronic F68 | 6.23 | 1.00 |
| PEG 400 | 71.73 | 11.47 |
| Water | 15.07 | 2.41 |
| BHT | 0.19 | 0.03 |
| Total | 625.26 | 100.0 |

### EXAMPLE 2

### Preparation of Liquid-Filled Capsules Containing 12.5 mg of Rifalazil

PEG-35 castor oil (Cremophor ELP), ascorbylpalmitate, Pluronic® F68, PEG 400, water, BHT, and rifalazil were mixed in proportions as provided below in Table 3. Capsules were filled with the liquid to produce liquid-filled capsules containing 12.5 mg of rifalazil each. The total fill weight per capsule was calculated based on target fill volume of 0.6 mL and density of 1.0421 g/mL.

TABLE 3
(Formulation B)

| Excipient | Amount (mg) per capsule | % (w/w) |
|---|---|---|
| Rifalazil (ABI-1648) | 12.5 | 2.00 |
| Ascorbyl Palmitate | 12.26 | 1.96 |
| Cremophor ELP | 508.77 | 81.37 |
| Pluronic F68 | 6.13 | 0.98 |
| PEG 400 | 70.59 | 11.29 |
| Water | 14.83 | 2.37 |
| BHT | 0.18 | 0.03 |
| Total | 625.26 | 100.0 |

### EXAMPLE 3

### Preparation of Liquid-Filled Capsules Containing 2.5 mg of Rifalazil

PEG-35 castor oil (Cremophor ELP), ascorbylpalmitate, PEG-6 apricot kernel oil (Labrafil M1944 CS), PEG-8 caprylic/capric glycerides (Labrasol), BHT, and rifalazil were mixed in proportions as provided below in Table 4. Capsules were filled with the liquid to produce liquid-filled capsules containing 2.5 mg of rifalazil each. The total fill weight per capsule was calculated based on target fill volume of 0.6 mL and density of 0.9911 g/mL.

TABLE 4
(Formulation C)

| Excipient | Amount (mg) per capsule | % (w/w) |
|---|---|---|
| Rifalazil (ABI-1648) | 2.5 | 0.42 |
| Ascorbyl Palmitate | 11.84 | 1.99 |
| Cremophor ELP | 148.04 | 24.89 |
| Labrafil M1944 CS | 296.08 | 49.79 |
| Labrasol | 136.02 | 22.87 |
| BHT | 0.18 | 0.03 |
| Total | 594.66 | 100.0 |

### EXAMPLE 4

### Preparation of Liquid-Filled Capsules Containing 12.5 mg of Rifalazil

PEG-35 castor oil (Cremophor ELP), ascorbylpalmitate, PEG-6 apricot kernel oil (Labrafil M1944 CS), PEG-8 caprylic/capric glycerides (Labrasol), BHT, and rifalazil were mixed in proportions as provided below in Table 5. Capsules were filled with the liquid to produce liquid-filled capsules containing 12.5 mg of rifalazil each. The total fill weight per capsule was calculated based on target fill volume of 0.6 mL and density of 0.9911 g/mL.

TABLE 5
(Formulation D)

| Excipient | Amount (mg) per capsule | % (w/w) |
|---|---|---|
| Rifalazil (ABI-1648) | 12.5 | 2.10 |
| Ascorbyl Palmitate | 11.64 | 1.96 |
| Cremophor ELP | 145.54 | 24.47 |
| Labrafil M1944 CS | 291.08 | 48.95 |
| Labrasol | 133.72 | 22.49 |
| BHT | 0.18 | 0.03 |
| Total | 594.66 | 100.0 |

### EXAMPLE 5

### Preparation of Liquid-Filled Capsules Containing 5 mg of Rifalazil

PEG-35 castor oil (3,102 g), Pluronic® F68 (44 g), PEG 400 (1,034 g), water (220 g) and rifalazil (30.743 g) were mixed, resulting in a volume of 4.058 L and a rifalazil concentration of 0.132 mL/mg. Capsules (fill weight of 0.66 g and a fill volume of 0.68 mL) were filled with the liquid to produce liquid-filled capsules containing 5 mg of rifalazil each.

### EXAMPLE 6

### Preparation of Rifalazil Formulations without a Surfactant Antioxidant

The excipients indicated for eac formulation and rifalazil were mixed in proportions as provided below in Tables 6-8. Capsules were filled with the liquid to produce liquid-filled capsules containing 12.5 mg of rifalazil each.

TABLE 6
(Formulation E)

| Excipient | Amount (mg) per capsule | % (w/w) |
|---|---|---|
| Rifalazil (ABI-1648) | 12.5 | 2.00 |
| Cremophor ELP | 519.34 | 83.06 |
| Pluronic F68 | 6.25 | 1.00 |
| PEG 400 | 72.14 | 11.52 |
| Water | 15.13 | 2.42 |
| Total | 625.26 | 100.00 |

TABLE 7
(Formulation F)

| Excipient | Amount (mg) per capsule | % (w/w) |
|---|---|---|
| Rifalazil (ABI-1648) | 12.5 | 2.00 |
| Cremophor ELP | 519.15 | 83.03 |
| Pluronic F68 | 6.25 | 1.00 |
| PEG 400 | 72.14 | 11.52 |
| Water | 15.13 | 2.42 |
| BHT | 0.18 | 0.03 |
| Total | 625.26 | 100.00 |

TABLE 8
(Formulation G)

| Excipient | Amount (mg) per capsule | % (w/w) |
|---|---|---|
| Rifalazil (ABI-1648) | 12.5 | 2.10 |
| Cremophor ELP | 145.51 | 24.47 |
| Labrafil M1944 CS | 291.56 | 48.93 |
| Labrasol | 145.51 | 24.47 |
| BHT | 0.18 | 0.03 |
| Total | 594.66 | 100.00 |

### EXAMPLE 7

### Rifalazil Solubility in Surfactants and Surfactant Mixtures

The solubility of rifalazil in various surfactants and surfactant mixtures was measured. Excess Rifalazil, drug substance, was equilibrated with the excipient or excipient blend at the indicated temperature for 24 hrs under constant mixing. At the end of the equilibration time, insoluble drug was removed by centrifugation and the supernatant was assayed by UV spectroscopy and/or HPLC to determine the concentration of Rifalazil. The components of formulations M1-M10 are provided in Table 9. The solubility data is provided in Table 10.

TABLE 9
Formulations of Rifalazil

| | Composition, % w/w | | | | | | | | | |
|---|---|---|---|---|---|---|---|---|---|---|
| Component | M1 | M2 | M3 | M4 | M5 | M6 | M7 | M8 | M9 | M10 |
| Etocas 35 NF | 85.03 | | 36.52 | 25 | 20 | 20 | | 35 | 40 | 23.48 |
| Cremophor ELP | | 85.03 | | | | | 25 | | | |
| Labrafil M1944 CS | | | 60 | 60 | 65 | 40 | 50 | 50 | 45 | 65 |
| Labrasol | | | | | | 20 | 25 | | | |
| Oleic Acid | | | | | | 20 | | | | |
| Pluronic F68 | 1 | 1 | 1 | | | | | 1 | 1 | |
| PEG 400 | 11.52 | 11.52 | | | | | | 11.52 | 11.52 | 11.52 |
| PEG 1500 | | | | 12.52 | | | | | | |
| TPGS | | | | | 12.52 | | | | | |
| Water | 2.42 | 2.42 | 2.42 | 2.42 | 2.42 | | | 2.42 | 2.42 | |

TABLE 10

| | Solubility | |
|---|---|---|
| Solvent | (mg/ml) | SD^{c} |
| Cremophor ELP | 35^{a} | 0.5 |
| Cremophor RH40 | 23^{a} | 1.0 |
| Labrafil M 1944 CS | 41.6 | 6.97 |
| Labrasol | 23 | NA^{d} |
| Lauroglycol 90 | 40 | NA |
| Oleic Acid | 59 | 8.7 |
| Peceol | 14 | NA |
| PEG 400 | 8 | 1.8 |
| PEG 1500 | 62^{b} | 0.5 |
| TPGS | 22^{a} | 5.3 |
| Labrafil M 19944 CS:Etocas 35 (5:1) | 29 | 3.2 |
| Formulations A and B | 23 | NA |
| Formulations C and D | 21 | NA |
| Formulation M1 | 15.51 | 0.97 |
| Formulation M2 | NA | NA |
| Formulation M3 | 25.82 | 4.47 |
| Formulation M4 | 34.08 | 1.84 |
| Formulation M5 | 28.35 | 2.20 |
| Formulation M6 | 30.84 | 3.71 |
| Formulation M7 | NA | NA |
| Formulation M8 | 26.87 | 1.90 |
| Formulation M9 | 19.62 | 2.29 |
| Formulation M10 | 36.90 | 0.34 |

^{a}upon equilibration at 37° C.;
^{b}upon equilibration at 60° C. to melt PEG 1500;
^{c}Standard Deviation;
^{d}NA = Not Available.

### EXAMPLE 8

### Rifalazil Stability in Various Formulations

The stability of rifalazil in various liquid-filled capsule formulations of the invention were measured as a function of storage conditions. Following storage under set conditions, each capsule was cut open using a clean razor blade and the contents dissolved in methanol, sonicate the contents for 5-10 minutes, rinsed, and diluted to a final concentration of about 0.1 mg/mL. The solution was assayed by reverse phase HPLC (Wavelength: 635 nm and 230 nm; Flow: 1.0 mL/min; Run time: 25 minutes; Mobile Phases: (A) 25 mM pH 5.5 Phosphate Buffer, (B) Methanol; linear gradient (% A/% B, minutes): (25/75,0), (5/95,20), (25/75,20.5), (25/75,25); Injection volume: 20 μL). The relative retention time of rifalazil N-oxide is 0.47 (rifalazil=1.0). The amount of N-oxide impurity present in each sample was assessed by comparison to a known standard. The results are provided in Table 11.

TABLE 11

| Formulation^{a} & | | | | |
|---|---|---|---|---|
| Antioxidants | Storage | Rifalazil N-oxide^{a} (%) | | |
| Present | Time | Initial | 25° C./60% RH | 40° C./75% RH |
| Formulation E | 2 weeks | NA^{c} | 0.27 | 0.35 |
| (No antioxidants) | | | | |
| Formulation F | 4 weeks | NA | 0.14 | 0.29 |
| (BHT only) | | | | |
| Formulation G | 4 weeks | NA | 0.37 | 0.94 |
| (BHT only) | | | | |
| Formulation B | 0 | ND^{b} | — | — |
| (BHT + Ascorbyl | 2 weeks | — | ND | ND |
| Palmitate) | 4 weeks | — | ND | ND |
| Formulation D | 0 | ND | — | — |
| (BHT + Ascorbyl | 2 weeks | — | 0.03 | 0.02 |
| Palmitate) | 4 weeks | — | 0.05 | ND |

^{a}All formulations incorporate 12.5 mg of Rifalazil per capsule
^{b}ND = Not Detected;
^{c}NA = Not available

The amount of BHT utilized in formulations F and G, where BHT is the only antioxidant present, is limited to 0.03% (w/w) due to the toxicity of this antioxidant. As a result, the protective effect in these formulations is limited unless another antioxidant, such as, ascorbyl-palmitate is included.

### EXAMPLE 9

### Pharmacokinetics of the Liquid-Filled Capsule Underfed and Fasted Conditions

Pharmacokinetic parameters were determined following a single peroral administration of 5 mg of rifalazil in healthy male beagle dogs. The rifalazil was formulated either as a liquid-filled capsule of Example 5 or as a powder-filled capsule containing microgranulated rifalazil as described in U.S. Pat. No. 5,547,683.

Both formulations were administered under fed and fasted conditions. All animals were fasted overnight prior to dosing. Animals designated as “fed” were administered a blended combination of dog chow and water in a 1:3 ratio (e.g. 250 g chow and 750 g water) via oral gavage at a dose volume of 20 mL/kg within approximately 30 minutes prior to dosing and food was provided Ad-libitum after approximately 4 hours following dosing. Animals in “fasted” groups were not fed prior to dosing and food was withheld until after approximately 4 hours after dosing.

Plasma samples (5.0 mL in EDTA tubes) for determination of rifalazil concentrations in plasma were obtained at hour: 0 (pre-dose) and at hours: 1.0, 2.0, 3.0, 4.0, 6.0, 8.0, 10.0, 12.0, 24, 36, 48, 72, 96, 168, 216 (Day 10), 336 (Day 15), 420, and 504 (Day 21), after administration of the rifalazil in either of the dosage forms.

Pharmacokinetic endpoints and parameters were calculated by noncompartmental analysis (NCA) using WinNonlin®.

The pharmacokinetic parameters Tₘₐₓ, Cₘₐₓ, AUCₜ, AUC_{∞}, T_{1/2} (elimination), and F (bioavailability) were calculated as well as the coefficient of variation (CV) in each. The results are provided in Table 11. 100% bioavailability was determined by comparision to the pharmacokinetic profile observed for intravenously administered rifalazil.

TABLE 11

| | Micro- | | Micro- | |
|---|---|---|---|---|
| | granulated | Liquid-filled | granulated | Liquid-filled |
| PK parameter | fasted | fasted | fed | fed |
| Tₘₐₓ (h) | 6.31 ± 7.11 | 1.87 ± 0.33 | 2.69 ± 0.92 | 2.33 ± 0.50 |
| Cₘₐₓ (ng/mL) | 27.2 ± 24.6 | 96.5 ± 18.2 | 52.8 ± 30.5 | 95.8 ± 33.6 |
| AUC_{∞} | 685 ± 359 | 1400 ± 266 | 830 ± 438 | 1420 ± 274 |
| (ng/mL × hr) | | | | |
| T_{1/2} (h) | 52.2 ± 17.3 | 46.8 ± 15.7 | 42.1 ± 14.8 | 43.4 ± 15.8 |
| F (%) | 26 | 53 | 32 | 54 |
| CV Cₘₐₓ (%) | 90.4 | 18.1 | 57.8 | 35.1 |
| CV AUC_{∞} | 53.4 | 19.2 | 52.7 | 19.3 |
| (%) | | | | |

The liquid-filled capsules of rifalazil exhibit a surprising increase in Cₘₐₓ under both fed (1.8 fold increase) and fasted (3.5 fold increase) conditions and an increase in AUC_{∞} under both fed (1.7 fold increase) and fasted (2.0 fold increase) conditions in comparison to microgranulated rifalazil.

The liquid-filled capsules of rifalazil also exhibit a surprising increase in bioavailability under both fed (1.7 fold increase) and fasted (2.0 fold increase) conditions in comparison to microgranulated rifalazil.

A comparison of the fed and fasted data obtained for the liquid-filled capsule formulation, i.e., AUC_{∞} (1400 vs. 1420) and Cₘₐₓ (96.5 vs. 95.8), shows no change in PK behavior, e.g., no “food effect.” In contrast, the microgranulated rifalazil exhibits a large food effect as demonstrated by the differences in AUC_{∞} (685 vs. 830) and Cₘₐₓ (27.2 vs. 52.8) under fed and fasted conditions.

A reduction in the coefficient of variation in Cₘₐₓ in both fed (1.6 fold decrease) and fasted (4.8 fold decrease) animals and a reduction in the coefficient of variation in AUC_{∞} in both fed (2.7 fold increase) and fasted (2.7 fold increase) animals is observed for the liquid-filled capsule in comparison to the microgranulated formulation.

Compositions of the invention can behave in a similar fashion with respect to Cₘₐₓ, AUC_{∞}, and bioavailability.

Changes in the formulation had no effect upon the elimination half-life (T_{1/2}) of rifalazil.

### EXAMPLE 10

### Rifalazil Formulations

The formulations of Tables 12-19 were prepared as described above. These formulations can be used in the methods, kits, and compositions of the invention.

TABLE 12

| Excipient | % (w/w) |
|---|---|
| Rifalazil (ABI-1648) | 2.10 |
| Lauroglycol 90 | 50.0 |
| Cremophor EL | 47.9 |
| Total | 100.00 |

TABLE 13

| Excipient | % (w/w) |
|---|---|
| Rifalazil (ABI-1648) | 2.10 |
| Lauroglycol 90 | 50.0 |
| Cremophor EL | 47.8 |
| Vitamin A Palmitate | 0.1 |
| w/dl-α-tocopherol | |
| Total | 100.00 |

TABLE 14

| Excipient | % (w/w) |
|---|---|
| Rifalazil (ABI-1648) | 2.10 |
| Cremophor RH40 | 47.9 |
| Lauroglycol 90 | 50.0 |
| Total | 100.00 |

TABLE 15

| Excipient | % (w/w) |
|---|---|
| Rifalazil (ABI-1648) | 2.10 |
| Ascorbyl Palmitate | 1.9 |
| Labrafil M 1944 CS | 50.0 |
| Cremophor EL | 45.9 |
| dl-α-tocopherol, | 0.1 |
| Vitamin E | |
| Total | 100.00 |

TABLE 16

| Excipient | % (w/w) |
|---|---|
| Rifalazil (ABI-1648) | 2.10 |
| Labrafil M 1944 CS | 50.0 |
| Cremophor EL | 47.80 |
| Vitamin A Palmitate | 0.1 |
| w/dl-α-tocopherol | |
| Total | 100.00 |

TABLE 17

| Excipient | % (w/w) |
|---|---|
| Rifalazil (ABI-1648) | 2.50 |
| Lauroglycol 90 | 52.02 |
| Cremophor EL | 45.24 |
| Vitamin A Palmitate | 0.24 |
| w/dl-α-tocopherol | |
| Total | 100.00 |

TABLE 18

| Excipient | % (w/w) |
|---|---|
| Rifalazil (ABI-1648) | 2.50 |
| TPGS | 19.50 |
| Cremophor RH40 | 29.01 |
| Lauroglycol 90 | 48.75 |
| Vitamin A Palmitate | 0.24 |
| w/dl-α-tocopherol | |
| Total | 100.00 |

TABLE 19

| Excipient | % (w/w) |
|---|---|
| Rifalazil (ABI-1648) | 2.50 |
| Ascorbyl Palmitate | 1.95 |
| Cremophor EL | 45.24 |
| Lauroglycol 90 | 50.21 |
| dl-α-tocopherol, | 0.1 |
| Vitamin E | |
| Total | 100.00 |

### OTHER EMBODIMENTS

All publications, patent applications, and patents mentioned in this specification are herein incorporated by reference.

While the invention has been described in connection with specific embodiments, it will be understood that it is capable of further modifications. Therefore, this application is intended to cover any variations, uses, or adaptations of the invention that follow, in general, the principles of the invention, including departures from the present disclosure that come within known or customary practice within the art.

Other embodiments are within the claims.

What we claim is:

1. A pharmaceutical composition for oral administration in unit dosage form comprising rifalazil, one or more surfactants, and a lipophilic antioxidant, wherein said one or more surfactants are from 20% to 99% (w/w) of said composition.

2. The pharmaceutical composition of claim 1, wherein said one or more surfactants are from 75% to 95% (w/w) of said composition.

3. The pharmaceutical composition of claim 1, wherein said lipophilic antioxidant is selected from carotenoids, tocopherols and esters thereof, tocotrienols and esters thereof, retinol and esters thereof, ascorbyl esters, butylhydroxyanisole (BHA), butylhydroxytoluene (BHT), propyl gallate, and mixtures thereof.

4. The pharmaceutical composition of claim 1, wherein said lipophilic antioxidant is an antioxidant surfactant.

5. The pharmaceutical composition of claim 4, wherein said antioxidant surfactant is retinyl palmitate, ascorbyl palmitate, or tocopheryl-PEG-1000-succinate.

6. The pharmaceutical composition of claim 1, wherein said composition comprises from 1 to 50% (w/w) of a first lipophilic antioxidant selected from retinol, retinyl palmitate, ascorbyl palmitate, tocopherol, tocotrienol and tocopheryl-PEG-1000-succinate and less than 0.1% (w/w) of a second lipophilic antioxidant selected from tocopherol, tocopherol acetate, tocopherol nicotinoate, tocopherol succinate, tocotrienol, tocotrienol acetate, tocotrienol nicotinoate, tocotrienol succinate, carotenoids, BHT, BHA, and propylgallate.

7. The pharmaceutical composition of claim 6, wherein said composition comprises from 1 to 20% (w/w) of said first lipophilic antioxidant.

8. The pharmaceutical composition of claim 1, further comprising a hydrophilic co-solvent selected from alcohols, polyethylene glycols, and mixtures thereof.

9. The pharmaceutical composition of claim 8, wherein said hydrophilic co-solvent is an alcohol selected from ethanol, propylene glycol, glycerol, and mixtures thereof.

10. The pharmaceutical composition of claim 8, wherein said hydrophilic co-solvent is a polyethylene glycol with a molecular weight of between 200 and 10,000 Da.

11. The pharmaceutical composition of claim 10, comprising PEG-35 castor oil.

12. The pharmaceutical composition of claim 11, comprising from 0.2 to 2.5% (w/w) rifalazil, from 75 to 85% (w/w) PEG-35 castor oil, from 0.5 to 1.5% (w/w) pluronic F68, from 8 to 15% PEG-400, from 1.5 to 2.5% (w/w) ascorbyl palmitate, from 0.01 to 0.05% (w/w) BHT, and from 1.5 to 2.5% (w/w) water.

13. The pharmaceutical composition of claim 1, comprising PEG-35 castor oil, PEG-8 caprylic/capric glycerides, and PEG-6 apricot kernel oil.

14. The pharmaceutical composition of claim 13, comprising from 0.2 to 2.5% (w/w) rifalazil, from 22 to 28% (w/w) PEG-35 castor oil, from 45 to 50% (w/w) PEG-6 apricot kernel oil, from 20 to 25% PEG-8 caprylic/capric glycerides, from 1.5 to 2.5% (w/w) ascorbyl palmitate, and from 0.01 to 0.05% (w/w) BHT.

15. The pharmaceutical composition of claim 9, wherein the solubility of said rifalazil in said one or more surfactants is greater than 16 mg/mL.

16. The pharmaceutical composition of claim 15, wherein the solubility of said rifalazil in said one or more surfactants is greater than 20 mg/mL.

17. The pharmaceutical composition of claim 1, wherein said unit dosage form comprises from between 1 and 30 mg of rifalazil.

18. The pharmaceutical composition of claim 1, wherein said one or more surfactants is present in an amount sufficient to produce, upon administration to fasted patients, a coefficient of variation in Cₘₐₓ of less than 60%.

19. The pharmaceutical composition of claim 1, wherein said one or more surfactants is present in an amount sufficient to produce, upon administration to fasted patients, a coefficient of variation in AUC_{∞} of less than 40%.

20. The pharmaceutical composition of claim 1, wherein said one or more surfactants is present in an amount sufficient to produce, upon administration to fasted patients, a mean bioavailability of greater than 30%.

21. A pharmaceutical composition for oral administration in unit dosage form comprising rifalazil and an antioxidant surfactant.

22. The pharmaceutical composition of claim 21, wherein said antioxidant surfactant is retinyl palmitate, ascorbyl palmitate, or tocopheryl-PEG-1000-succinate.

23. A pharmaceutical composition for oral administration in unit dosage form comprising rifalazil, a surfactant, and a lipophilic antioxidant, wherein said lipophilic antioxidant is present in an amount sufficient to reduce the oxidation of rifalazil.

24. The pharmaceutical composition of claim 23, wherein upon storage of said unit dosage form at 25° C. and 60% relative humidity for a period of one month, less than 0.2% of said rifalazil is converted to rifalazil N-oxide.

25. The pharmaceutical composition of claim 24, wherein upon storage of said unit dosage form at 25° C. and 60% relative humidity for a period of six months, less than 0.2% of said rifalazil is converted to rifalazil N-oxide.

26. The pharmaceutical composition of claim 25, wherein upon storage of said unit dosage form at 25° C. and 60% relative humidity for a period of twelve months, less than 0.2% of said rifalazil is converted to rifalazil N-oxide.

27. A method of treating a bacterial infection in a patient, said method comprising administering to said patient a pharmaceutical composition comprising rifalazil, 20% to 99% (w/w) of one or more surfactants, and a lipophilic antioxidant, wherein said composition is administered in an amount effective to treat said infection.

28. The method of claim 27, wherein said infection is selected from community-acquired pneumonia, upper and lower respiratory tract infection, skin and soft tissue infection, bone and joint infection, hospital-acquired lung infection, acute bacterial otitis media, bacterial pneumonia, complicated infection, noncomplicated infection, pyelonephritis, intra-abdominal infection, deep-seated abcess, bacterial sepsis, central nervous system infection, bacteremia, wound infection, peritonitis, meningitis, infections after burn, urogenital tract infection, gastro-intestinal tract infection, pelvic inflammatory disease, endocarditis, and intravascular infection.

29. The method of claim 27, wherein said pharmaceutical composition is administered for prophylaxis against an infection resulting from a surgical procedure or implantation of a prosthetic device.

30. The method of claim 27, wherein said bacterial infection is by Gram-positive bacterium.

31. The method of claim 27, wherein said bacterial infection is by multi-drug resistant bacteria.

32. The method of claim 27, wherein said bacterial infection is by Chlamydia pneumoniae or Chlamydia trachomatis.

33. A method of treating an atherosclerosis-associated disease in a patient diagnosed as having said disease, said method comprising administering to the patient (i) rifalazil and (ii) a lipophilic antioxidant simultaneously or within 14 days of each other in an amount, that together, is effective to treat said disease in said patient.

34. The method of claim 33, wherein said disease is atherosclerosis or peripheral artery disease.

35. A pharmaceutical composition comprising (i) rifalazil and (ii) a lipophilic antioxidant wherein said rifalazil and said a lipophilic antioxidant are each present in an amount that together is effective to treat an atherosclerosis-associated disease when administered to a patient.

36. The pharmaceutical composition of claim 35, wherein said disease is atherosclerosis or peripheral artery disease.

37. A kit, comprising:
(i) a composition comprising rifalazil and a lipophilic antioxidant; and
(ii) instructions for administering said composition to a patient diagnosed with an atherosclerosis-associated disease.

38. A kit, comprising:
(i) rifalazil; and
(ii) instructions for administering said rifalazil and a lipophilic antioxidant to a patient diagnosed with an atherosclerosis-associated disease.

39. The kit of claim 37 or 38, wherein said disease is atherosclerosis or peripheral artery disease.
